(19) United States
(12) Patent Application Publication
(10) Pub. No.: US 2023/0279419 A1
(43) Pub. Date: Sep. 7, 2023

(54) ENHANCEMENT OF PRODUCTIVITY IN C3 PLANTS

(71) Applicant: OXFORD UNIVERSITY INNOVATION LIMITED, Oxford (GB)

(72) Inventors: Ross HENDRON, Oxford (GB); Enrique LOPEZ-JUEZ, Egham (GB); Steven KELLY, Oxford (GB)

(73) Assignee: OXFORD UNIVERSITY INNOVATION LIMITED, Oxford (GB)

(21) Appl. No.: 17/999,358

(22) PCT Filed: May 18, 2021
(86) PCT No.: PCT/GB2021/051195

§ 371 Date: Nov. 18, 2022

(30) Foreign Application Priority Data
May 20, 2020 (GB) 2007526.3

(51) Int. Cl. C12N 15/82; C07K 14/415

(52) U.S. Cl. CPC C12N 15/8269; C07K 14/415; C12N 15/8225

(57) ABSTRACT

Vascular sheath tissue-specific expression of phytochrome B or variants thereof in C3 plants increases photosynthesis rate and/or introduces a carbon refixation mechanism. The heritable genetic material of a C₃ plant cell is altered such that one copy of phytochrome B, or active variant or functional fragment thereof is expressed specifically in vascular sheath cells. Whole plants are regenerated from these genetically altered plant cells. Alternatively, a Crispr modification of a native phytochrome locus in a plant cell is used to insert a vascular sheath-specific regulatory element, e.g. promoter or enhancer element, so that phytochrome B is expressed in vascular sheath cells of a regenerated whole plant. Genetically altered whole plants have increased yield-related traits, e.g. increased seed yield, resulting from the enhancement of photosynthesis and/or introduction of a carbon refixation mechanism.

### FIELD OF THE INVENTION

The present invention relates generally to the field of plant molecular biology and concerns a method for tissue specific expression of a certain gene or genes which enhance yield-related traits in plants by increases in photosynthesis. The invention concerns expression constructs useful in the methods of the invention. The invention also concerns genetically altered plants which have increased yield-related traits resulting from the enhancement of photosynthesis. The invention further concerns parts of such altered plants, such as plant cells, plant parts, plant organs, fruits, seeds, embryos, germplasm and processed plant products.

### INCORPORATION BY REFERENCE

Each patent, publication, and non-patent literature cited in the application is hereby incorporated by reference in its entirety as if each was incorporated by reference individually.

### BACKGROUND

Phytochrome B (PHYB) is a red/far-red photoreceptor involved in the regulation of multiple plant processes including germination, de-etiolation, light-mediated plant development (photomorphogenesis), flowering, responses to shade, and chloroplast biogenesis. PHYB also regulates temperature responses by associating with the promoters of key target genes in a temperature-dependent manner and subsequently repressing their expression. PHYB may act as a thermal timer that integrates temperature information over the course of the day/night cycle.

PHYB exists in two inter-convertible forms: Pr (inactive in the dark) and Pfr (active in the light). Active Pfr PHYB accumulates in the nucleus after exposure to red light where it functions to initiate multiple regulatory cascades that control the aforementioned plant processes. There is a constitutively active variant of PHYB known as YHB. This variant it contains a single amino acid change from Y to H at site 276 in the Arabidopsis version of the PHYB gene. YHB performs the same regulatory functions as active PHYB but does not require light to be activated. Throughout this application the term “active variant” in reference to PHYB refers to all constitutively active variants of PHYB and includes YHB.

Due to its regulatory role in multiple plant processes, all previous manipulations of PHYB or YHB have resulted in developmental defects that make manipulation of the timing or location of expression of this gene unsuitable for improving crops. Repeatedly observed defects arising from manipulation of PHYB or YHB expression include dwarfism, delayed flowering time, thicker leaves, smaller tubers (in potatoes), decreased water use efficiency, and increased drought susceptibility. Moreover, no increase in photosynthetic rate has been demonstrated in plants over expressing PHYB or YHB when rates are normalized for increased nitrogen investment.

Most of the PHYB regulated processes found in Arabidopsis are also regulated by PHYB in other plant species, e.g. germination, de-etiolation, light-mediated plant development (photomorphogenesis), flowering, responses to shade, and chloroplast biogenesis. Also, many plants have genes encoding multiple orthologs of PHYB. The genomes of flowering plants also have genes encoding other phytochromes, such as phytochrome A (PHYA) whose gene product has an antagonistic relationship with PHYB, often promoting opposing effects, e.g. in the shade tolerance response. Plants that overexpress PHYA also have effects that are deleterious to plant productivity.

The following is a list of examples where over-expression of PHYB or YHB (or other related phytochrome genes) resulted in effects that were deleterious to the productivity of plants:

Wagner et al., (1991) “Overexpression of Phytochrome B induces a short hypocotyl phenotype in transgenic Arabidopsis” Plant Cell. 3(12): 1275-1288. This describes how the systemic overexpression of native PHYB in Arabidopsis plants or rice PHYB in Arabidopsis plants alters photomorphogenesis resulting in shortened hypocotyls and shorter plants.

Thiele et al., (1999) “Heterologous Expression of Arabidopsis Phytochrome B in Transgenic Potato Influences Photosynthetic Performance and Tuber Development” Plant Physiology. 120: 73-81. This describes overexpression of PHYB in potato. This was found to cause a variety of negative changes to the plants. There was a delay in flowering time, increased branching, a higher number of smaller and thicker leaves due to larger mesophyll cells, and a deceleration of chlorophyll degradation. There was no difference between plants overexpressing PHYB and wild type plants in terms of carbon dioxide fixation when fixation rates were normalized per unit of chlorophyll. Modified plants were also found to have negative effects such as smaller tubers and a delay in tuber formation such that the yield of modified plants was lower than that of unmodified control plants in the same growing conditions.

Rao et al., (2011) “Overexpression of the phytochrome B gene from Arabidopsis thaliana increases plant growth and yield of cotton (Gossypium hirsutum)” J. Zheijiang Univ. Sci. B. 12: 326-334. This describes how overexpression of PHYB in cotton gave faster growth, however it also caused numerous negative effects such as quadrupling of transpiration rate (i.e. making the plant more drought susceptible and less water use efficient), dwarfism, thicker leaves and decreased apical dominance resulting in more branching.

Halliday et al, (1997) “Expression of heterologous phytochromes A, B or C in transgenic tobacco plants alters vegetative development and flowering time” The Plant Journal 12: 1079-1090. This describes overexpression of PHYB in tobacco resulting in the negative effects of delayed flowering and dwarfing.

Husaineid et al., (2007) “Overexpression of homologous phytochrome genes in tomato: exploring the limits in photoperception” J. Exp. Bot. 58: 615-626. This describes tomato lines overexpressing PHYA, PHYB1, or PHYB2, under control of the constitutive double-35S (CaMV) promoter. This resulted in the negative effects of dwarfing and greater anthocyanin production.

Holefors et al., (2000) “The Arabidopsis phytochrome B gene influences growth of the apple rootstock M26” Plant Cell Reports 19: 1049-1056. This describes over expression of PHYB in Apple rootstock M26 (Malus domestica). This resulted in the negative effects of reduction in stem length, as well as reduction in shoot, root and plant dry weights.

Distefano et al., (2013) “Ectopic expression of Arabidopsis Phytochrome B in Troya citrange affects photosynthesis and plant morphology.” Scientia Horticulturae 159:1-7. This describes how overexpression of PHYB in citrus increased expression of photosynthesis genes and leaf chlorophyll content but also increased stomata density, altered branch angles and lowered photosynthesis rates.

Zheng et al., (2001) “Modification of Plant Architecture in Chrysanthemum by Ectopic Expression of the Tobacco Phytochrome B1 Gene” J. Am. Hort. Soc. Sci. 126(1): 19-26. This describes ectopic expression of tobacco PHYB1 gene in Chrysanthemum under control of the CaMV 35S promoter. The resulting plants exhibited negative effects such as shorter stature with larger branch angles than wild-type plants. The effect of the PHYB1 expression was comparable to commercial growth retardants and thus the authors suggest is that an application of PHYB1 overexpression might be an alternative to the application of exogenous growth retardants.

Yang et al., (2013) “Deficiency of Phytochrome B alleviates chilling-induced photoinhibition in rice” Am. J. Bot. 100(9): 1860-1870. This describes how mutant rice plants that had reduced PHYB expression were less photoinhibited than wildtype plants during and following chilling stress, and had measurably higher photosystem II efficiency and chlorophyll content than wildtype control plants. Hence this work showed that reducing PHYB expression caused an enhancement of photosynthesis. These findings suggest that crop improvement should follow a strategy of reducing PHYB expression, rather than increasing it.

Su & Lagarias (2007) “Light-Independent Phytochrome Signaling Mediated by Dominant GAF Domain Tyrosine Mutants of Arabidopsis Phytochromes in Transgenic Plants. Phytochrome B-Y276H (YHB)” The Plant Cell, Vol 19: 2124-2139. This describes a mutant form of the Arabidopsis thaliana PHYB protein known as YHB in which the tyrosine (Y) at position 276 is converted to a histidine (H). The Y276H mutant is profluorescent and photoinsensitive. When YHB is expressed in plants a range of altered light signalling activities are found associated with this mutation resulting in small, dwarfed plants.

U.S. Pat. No. 8,735,555 B2 discloses mutant phytochromes which when introduced into Arabidopsis alter the photomorphogenic properties of the plant. A Y276H mutant of PHYB is described which in a plant is light-stable and results in an altered photomorphogenesis as compared to the same species or variety lacking the mutant. The transgenic plants expressing a mutant Y276H Arabidopsis phytochrome showed decreased shade avoidance as compared to the same species of plant lacking the mutant phytochrome, and had altered photomorphogenesis resulting in dwarfing.

Hu et al., (2019) “Regulation of monocot and dicot plant development with constitutively active alleles of phytochrome B.” Plant Direct, 4:1-19. This describes experiments in which either Arabidopsis YHB or rice YHB were overexpressed in Arabidopsis, rice, tobacco, tomato and Brachypodium. In all cases, a suite of developmental changes were induced which consistently resulted in altered plant architecture and reduced plant height. Moreover, both shoot branching and seed yield were negatively impacted by YHB overexpression in all of these species.

US 2004/0268443 A1 (Wu et al.) describes increasing the accumulation of a heterologous PHYA in a plant, such as, for example, a Basmati rice plant, to alter the plant architecture and thereby minimize or overcome the plant's shade-avoidance growth response. More particularly, the elite indica rice, Pusa Basmati-1 (“PBNT”) was transformed with the Arabidopsis PHYA under the control of a light-regulated, tissue-specific, rice RbcS promoter, resulting in a large number of independent transgenic lines. Results from the fifth generation (generation “T4”) homozygous transgenic lines showed high levels of PHYA accumulation in the leaves of light-grown plants and altered plant architecture compared to unmodified plants.

US 2005/0120412 A1 (Wallerstein) discloses a long day plant modified to overexpress a PHYA or PHYB protein in at least a portion of the cells of the plant, such that flowering shoots, flowering, flowers, seeds or fruits thereof develop under substantially shorter days than that required for development of corresponding said flowering-shoots, flowering pots, flowers, seeds or fruits in a similar unmodified long day plant. An expression cassette is provided comprising the phytochrome coding sequence under the control of a functional promoter. A Cauliflower Mosaic virus (CaMV) 35S promoter is used specifically.

CN 106854240 A (BIOTECHNOLOGY RES CENTER SHANDONG ACAD OF AGRICULTURAL SCIENCES) discloses the nucleotide sequence and amino acid sequence of the phytochrome AhphyB of peanut. The phytochrome AhphyB is proposed for regulating and controlling a high-irradiance reaction of shade avoidance. The AhphyB of peanut is expressed in Arabidopsis and the effect of light conditions on hypocotyl growth is tested. The proposal is to upregulate phyB expression so that peanut pod development can be controlled and high-yield peanut species can be grown in a corn and peanut intercropping mode.

WO 2005093054 A1 (KANSAI TECH LICENSING ORG) discloses how the N-terminal region of the phytochrome molecule has intranuclear signal transduction ability. A N-terminal fragment of phytochrome fused with a domain involved in the quantification and a nuclear localization signal has a photosensitivity that is 100 times or more higher than that of the full-length phytochrome molecule. This artificial phytochrome molecule is used to modify plants, e.g. rice, in order to enhance photosensitivity, resulting in an increase in pigment, prolongation of flowering period, enlargement of ovary, or an enlargement of stems.

WO 99/31242 A1 (KWS) concerns plants which overexpress phytochrome B by introducing or activating a phytochrome B gene in the plant. A chimeric Arabidopsis thaliana phyB gene was transformed into potato plants via Agrobacterium tumefaciens-mediated gene transfer. Transgenic plants that express the phytochrome B from Arabidopsis exhibit dwarfism, reduced apical dominance, and darker green leaves. Various phenotypic changes appeared to correlate to increased photosynthetic output. An increased number and yield of tubers was found in transformed plants. Transformation of potato with a phytochrome b from Solanum tuberosum can also improve properties of the plants, although it improves a fewer number of traits than the gene from Arabidopsis thaliana.

US2007295252A1 (Dasgupta) discloses nucleic acid molecules identified from Zea mays such as promoters, leaders and enhancers, as well as combinations of said regulatory elements in chimeric molecules. The regulatory elements identified are from fructose 1-6 bisphosphate aldolase (FDA), pyruvate orthophosphate dikinase (PPDK), or ribulose bisphosphate carboxylase activase (RCA) genes. The regulatory element molecules preferably modulate transcription of genes in leaf tissue. The regulatory elements include promoters, enhancers, leaders, and combinations of such regulatory elements in the form of chimeric or hybrid expression elements. Transgenic maize plants and seeds containing the DNA constructs, comprising a promoter and regulatory elements operably linked to a heterologous DNA molecule are described, and whereby the transgenic plant expresses an agronomically desirable phenotype.

CN108913717A (UNIV HENAN) discloses Crispr-Cas9 based rice phytochrome PHYB gene editing vector. The vector is used to mutate the rice phytochrome PHYB gene without mutation of other genes in the plant. Four mutant phyB mutants were created in rice which are then screened for agronomically useful traits. The gene editing vector simplifies the workload of creating phyB mutants and makes the process of creating mutants more controllable.

Ganesan et al. (2017) “Development of transgenic crops based on photo-biotechnology” Plant Cell Environ. 40: 2469-2486 is a review article which looks generally at modulation of photoreceptors. Various attempts involving modulation of PHYB are referred to (also listed above), but all give rise to results that are undesirable in terms of plant growth and development and negatively impact on plant productivity.

In summary, despite many attempts at manipulating PHYB, YHB, and PHYA expression in plants, none of the aforementioned patent disclosures has succeeded in improving photosynthesis, growth, and yield. Instead, they have negatively affected plant development, plant architecture, and water use efficiency. The difficulty is that phytochromes have a central regulatory role in all plants and all previous manipulations of these genes have resulted in developmental defects that make manipulation of the timing or location of expression of this gene unsuitable for improving crops.

Leegood, R. C. (2008) “Roles of the bundle sheath cells in leaves of C₃ plants” J. Exp. Bot. vol 59 pp 1663-1673 is a review article which explains the structure and functions of the bundle sheath cells that surrounds the veins in the leaves of many C₃ plants. Although it is clear that the cells of the bundle sheath and their extensions have a number of metabolic roles, for example, in synthesis and storage of carbohydrates, the uptake, metabolism, and mobilization of nitrogen and sulphur, and in antioxidant metabolism, it is clear that much more needs to be known about their activities in the leaves of C₃ plants.

### BRIEF SUMMARY OF THE DISCLOSURE

The inventors have discovered that if a gene of interest (GOI), particularly PHYB, is expressed predominantly in the bundle sheath cells of plants compared to other plant cells or tissues, then this leads to a range of wholly beneficial traits and no detrimental traits in terms of plant growth, development, and productivity.

Accordingly, the present invention provides a method of increasing the photosynthetic capacity of a C₃ plant, the method comprising altering the heritable genetic material of the plant such that a GOI is expressed in one or more of the vascular sheath cells of the plant, and wherein the GOI is expressed under the control of a gene expression regulatory element active in vascular sheath cells of the plant.

As will be readily understood by a person of skill in the art, the methods of the invention are for providing C₃ plants with an altered genetic make-up, compared to normal or wild-type plants, or any plants which have not been subjected to a method of the invention. There are now many ways in which the genome of a plant can be altered, and various terms are used to describe these. Each of these terms will be familiar to the skilled reader and include “genetically modified”, “genetically engineered” or “gene edited” and are often used interchangeably. All refer to a plant which has had its genome sequence altered with respect to a non-modified control plant. This alteration could be caused by insertion of one or more polynucleotides of the invention into the genome of the target plant though any transformation, transfection, transduction, or genome engineering technique. This alteration may also be caused by nuclease-mediated genome editing, prime editing, and/or base editing.

In embodiments of methods of the invention as herein defined, the genetic material of cells of a plant are preferably first altered and then a genetically altered whole plant is regenerated from the genetically altered cell(s). The regeneration of plants from cells or plant tissues is something which will be familiar to a person of skill the art from the established literature.

In preferred methods, the gene expression regulatory element is active specifically in at least some of the vascular sheath cells of the plant, whereby the GOI under the control of the regulatory element is expressed specifically in at least some of the vascular sheath cells of the genetically altered whole plant. The term “specific” as used herein may also include the meanings of “exclusive” or “strongly preferential”.

Additionally or alternatively, the GOI is phytochrome B, or an active variant thereof, or functional fragment, as is further defined hereinafter.

An altering of the heritable genetic material may comprise inserting a polynucleotide into the heritable genetic material of a cell of the plant.

In some methods, the altering of the heritable genetic material may comprise introducing a gene repair oligonucleobase (GRON)-mediated mutation into a target DNA sequence of the heritable genetic material of a cell of the plant. In further methods, the cell of a plant may be exposed to a DNA cutter and a GRON. The DNA cutter may comprise a meganuclease, a transcription activator-like effector nuclease (TALEN), a zinc finger, an antibiotic, or a Cas protein.

The altering of the heritable genetic material may comprise using zinc finger nucleases (ZNFs) and/or transcription activator-like effector nucleases (TALENs) for site-specific homologous recombination of the heritable genetic material of a cell of the plant. Thus, the invention provides methods of altering the genetic material of plants which carry out C₃ photosynthesis in at least parts thereof, the alteration being such that the modified plants express PHYB, or active variant such as YHB, or functional fragment thereof in at least some; optionally all, of the vascular sheath cells of the plant. This expression in vascular sheath cells is additional to the normal expression patterns of at least one copy of the PHYB gene in the plant. As will be appreciated, at least one copy of PHYB and accompanying expression control elements preferably remains unaltered so that the growth and development of the modified plant may be substantially unchanged compared to unmodified plant of same genotype.

A method in accordance with the invention may employ classical and well-known techniques of genetic modification, involving a method of transformation, whereby one or more additional copies of a native or exogenous PHYB gene, active variant, or functional fragment thereof, can be incorporated into a plant genome, together with the necessary vascular sheath cell expression regulatory element(s). Such incorporation is preferably stable and heritable so as to permit introduction of the modification into particular lines of crop plants; advantageously for the purposes of crop improvement or breeding programmes. Also, as already noted above, a CRISPR-Cas gene modification method may be used, whereby a guide RNA (gRNA) is chosen to target the action of a CRISPR associated protein (Cas) to a desired genomic locus resulting in a homologous recombination (HR) event, i.e. insertion-deletion of a desired polynucleotide into the plant genome.

In some embodiments, a method of the invention may involve simply introducing the vascular sheath expression regulatory element, such as a promoter sequence or DNA regulatory element, into position upstream of an existing native PHYB coding gene sequence in the genome, by any number of gene editing approaches. In operating such embodiments of the invention, a guided approach is convenient, for example, using a CRISPR associated protein (Cas) which can be directed by a gRNA, or any other genome editing nucleases (ZFNs, TALENs and other Cas proteins), to cleave specific genomic regions and introduce the necessary polynucleotide as a repair DNA template by homologous recombination.

In accordance with an aforementioned method of the invention involving CRISPR-Cas, the one or more polynucleotides used to transform plant material may include a polynucleotide encoding a Cas protein, optionally also a guide RNA (gRNA), wherein the gRNA directs the Cas protein to the locus of at least one copy of an endogenous PHYB gene in the plant cell genome, whereby the regulatory element is inserted so as to cause expression of the endogenous copy or copies of the PHYB specifically in at least some of the vascular sheath cells of the regenerated plant.

In some embodiments, the gRNA is synthesized as a single guide RNA (sgRNA) or as a CRISPR-RNA (crRNA): trans-activating CRISPR RNA (tracrRNA) duplex. In some embodiments, multiple gRNAs, crRNAs, or tracrRNAs may be used simultaneously, for example, to target multiple genomic regions. In some embodiments, different types of CRISPR-Cas systems and orthogonal Cas proteins mat be used simultaneously.

As used herein, the term “Cas” or “Cas protein” or “CRISPR-Cas protein” or “Cas nuclease” or “Cas moiety” or “Cas domain” refers to a CRISPR associated protein, including any equivalent or functional fragment thereof and any Cas homolog, ortholog, or paralog from any organism, and any mutant or variant of a Cas, naturally-occurring or engineered. The CRISPR-Cas protein can be, for example, Cas9, Cas12a, or Cas12b. The CRISPR endonucleases can be produced using E. coli expression systems. For example, encoding a Cas gene driven by the T7 promoter into E. coli is one mechanism. CRISPR-Cas proteins may also include Cas12c (or C2c3), Cas 12d (or CasY), Cas12e (or CasX), Cas13a (or C2c2), Cas13b (or C2c6), Cas13(c) or C2c7, Cas 13d (or Casrx), or a functional fragment thereof.

As used herein, the term “Cas9” or “Cas9 nuclease” or “Cas9 moiety” or “Cas9 domain” or “Csn1” refers to a CRISPR associated protein 9, or functional fragment thereof, and embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. More broadly, a Cas9 is a type of “RNA-programmable nuclease” or “RNA-guided nuclease” or more broadly a type of “nucleic acid programmable DNA binding protein (napDNAbp)”. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the evolved base editors of the invention.

As used herein, the term “Cas12a” or “Cas12a nuclease” or “Cas12a moiety” or “Cas12a domain” is used interchangeably with Cpfl. The term “Cas12a” and may also comprise a CRISPR associated protein 12a, or functional fragment thereof, and embraces any naturally occurring Cas12a from any organism, any naturally-occurring Cas12a equivalent or functional fragment thereof, any Cas homolog, ortholog, or paralog from any organism, and any mutant or variant of a Cas12a, naturally-occurring or engineered. This extends to orthologs of Cas12a, as well as polynucleotide sequences encoding such orthologs or systems and vectors or vector systems comprising such and delivery systems comprising such. More broadly, a Cas12a is a type of “RNA-programmable nuclease” or “RNA-guided nuclease” or more broadly a type of “nucleic acid programmable DNA binding protein (napDNAbp)”. The term Cas12a is not meant to be particularly limiting and may be referred to as a “Cas12a or equivalent.” Exemplary Cas12a proteins are further described herein and/or are described in the art and are incorporated herein by reference.

As used herein, the term “Cas12b” or “Cas12b nuclease” or “Cas12b moiety” or “Cas12b domain” is used interchangeably with C2c1 or Cpf2. The term “Cas12b” and may also comprise a CRISPR associated protein 12b, or functional fragment thereof, and embraces any naturally occurring Cas12b from any organism, any naturally-occurring Cas12b equivalent or functional fragment thereof, any Cas homolog, ortholog, or paralog from any organism, and any mutant or variant of a Cas12b, naturally-occurring or engineered. This extends to orthologs of Cas12b, as well as polynucleotide sequences encoding such orthologs or systems and vectors or vector systems comprising such and delivery systems comprising such. More broadly, a Cas12b is a type of “RNA-programmable nuclease” or “RNA-guided nuclease” or more broadly a type of “nucleic acid programmable DNA binding protein (napDNAbp)”. The term Cas12b is not meant to be particularly limiting and may be referred to as a “Cas12b or equivalent.” Exemplary Cas12b proteins are further described herein and/or are described in the art and are incorporated herein by reference.

As noted above, a method in accordance with the invention may employ emerging techniques of genetic modification, as well. For example, techniques may involve introducing a gene repair oligonucleobase (GRON)-mediated mutation into a target deoxyribonucleic acid (DNA) sequence in a plant cell, as described and elaborated on in U.S. Pat. No. 9,957,515 B2. Techniques may also involve combining GRON-mediated mutations into a target DNA sequence in a plant cell in combination with other DNA editing or recombination technologies including, but not limited to, gene targeting using site-specific homologous recombination by zinc finger nucleases, Transcription Activator-Like Effector Nucleases (TALENs) or Clustered Regularly Interspaced Short Palindromic Repeats (CRISPRs). Techniques may also include exposing a plant cell to a DNA cutter (a moiety that effects a strand break) and a GRON. Nonlimiting examples of DNA cutters that may be used include meganucleases, TALENs, antibiotics, zinc fingers and CRISPRs or CRISPR/Cas systems.

Techniques may involve introducing a purified nuclease protein to a plant cell, without the need for inserting exogenous genetic material. These techniques may involve the techniques described in EP3008186B1. In particular, the techniques may involve providing a plant cell that comprises an exogenous gene to be modified; providing a Cas9 endonuclease protein targeted to the endogenous gene; and transfecting the plant cell with said Cas9 endonuclease protein using biolistic or protoplast transformation, such that the Cas9 endonuclease introduces one or more double stranded DNA breaks (DSB) in the genome, to produce a plant cell or cells having a detectable targeted genomic modification without the presence of any exogenous Cas9 genetic material in the plant genome, as disclosed in EP3008186B1. Transfection can be effected through delivery of the sequence-specific nuclease into isolated plant protoplasts. For example, transfection can be effected delivery of the sequence-specific nuclease into isolated plant protoplasts using polyethylene glycol (PEG) mediated transfection, electroporation, biolistic mediated transfection, sonication mediated transfection, or liposome mediated transfection.

An RNA template may also be also be used. For example, another aspect of the invention is directed to a conjugate of CRISPR Cas protein-guide RNA complex(es), wherein the guide RNA(s) is a conjugate of a crRNA, dual guide RNAs, an sgRNA or an 1gRNA with one or more single strand DNAs (ssDNA) as a donor template for gene editing. Therefore, in accordance with an aforementioned method of the invention involving CRISPR-Cas, the one or more polynucleotides used to transform plant material may include a polynucleotide encoding a CRISPR-Cas protein, optionally also at least one guide RNA (gRNA), wherein the gRNA(s) direct the CRISPR-Cas protein to the locus of at least one copy of an endogenous phytochrome B in the plant cell genome, whereby the regulatory element is inserted so as to cause expression of the copy or copies of the phytochrome B specifically in at least some of the vascular sheath cells of the regenerated plant. The at least one copy which is inserted in the plant cell genome may be inserted using a viral vector-based system, In the context of genetic engineering, any reference to insertions or inserting a regulatory element may refer to any donor, donor sequence, or donor polynucleotide which is inserted into the plant cell genome, for example, using a system described above. Donor(s) (donor sequence(s), or donor polynucleotide(s)) may refer to polynucleotides, RNA, DNA, or genome insertions.

A sequence-specific nuclease to be delivered may be either in the form of purified nuclease protein, or in the form of mRNA molecules which can are translated into protein after transfection. Nuclease proteins may be prepared by a number of means known to one skilled in the art, using available protein expression vectors such as, but not limited to, pQE or pET. Suitable vectors permit the expression of nuclease protein in a variety of cell types (E. coli, insect, mammalian) and subsequent purification. Synthesis of nucleases in mRNA format may also be carried out by various means known to one skilled in the art such as through the use of the T7 vector (pSF-T7) which allows the production of capped RNA for transfection into cells. The mRNA may be modified with optimal 5′ untranslated regions (UTR) and 3′ untranslated regions. UTRs have been shown to play a pivotal role in post-translational regulation of gene expression via modulation of localization, stability and translation efficiency (Bashirullah A, Cooperstock R, Lipshitz H (2001) Spatial and temporal control of RNA stability. PNAS 98: 7025-7028). As noted above, mRNA delivery is desirable due to its non-transgenic nature; however, mRNA is a very fragile molecule, which is susceptible to degradation during the plant transformation process. Utilization of UTRs in plant mRNA transformations allow for increased stability and localization of mRNA molecules, granting increased transformation efficiency for non-transgenic genome modification.

In some embodiments, the CRISPR reagents may be delivered using Agrobacterium-mediated or particle bombardment-mediated transformation with DNA harbouring CRISPR expression cassettes. For example, in some embodiments, mRNA encoding Cas proteins can be co-delivered with the gRNA(s) into plants by particle bombardment. In other embodiments, the Cas protein and the gRNA(s) can be preassembled to form ribonucleoproteins (RNPs) and introduced into plants through a donor template. Delivery of RNPs into plants may be achieved through various methods. Methods include, for example, polyethylene glycol (PEG)-mediated cell transfection, particle bombardment, electroporation, and lipofection. The term “a donor template” refers to a transgene cassette or a gene-editing-sequence flanked with homologous regions to recombine with the host loci and replace the mutated DNA with the correct sequence by homologous gene repair (HDR)/single-strand DNA recombineering (SSDR). As used herein, a donor template may be referred to as a “donor polynucleotide.” A donor polynucleotide can be an ssDNA or a dsDNA or a plasmid/vector, and may be chemically conjugated to guide RNA(s) or Cas protein via a covalent linker. A donor template can be chemically synthesized and equipped with chemical functions for conjugations/ligations. A conjugating donor template may also be prepared by in vitro gene synthesis at the presence of a DNA polymerase, with chemical functions, e.g. an amine and an alkyne, enzymatically incorporated at its 5′ or 3′-end for chemical conjugation/ligation from a nucleoside triphosphate analogue.

Purified nucleases are delivered to plant cells by a variety of means. A sequence-specific nuclease to be delivered may be either in the form of purified nuclease protein, or in the form of mRNA molecules which can are translated into protein after transfection. Nuclease proteins may be prepared by a number of means known to one skilled in the art, using available protein expression vectors such as, but not limited to, pQE or pET. Suitable vectors permit the expression of nuclease protein in a variety of cell types (E. coli, insect, mammalian) and subsequent purification. Synthesis of nucleases in mRNA format may also be carried out by various means known to one skilled in the art such as through the use of the T7 vector (pSF-T7) which allows the production of capped RNA for transfection into cells. The mRNA may be modified with optimal 5′ untranslated regions (UTR) and 3′ untranslated regions. UTRs have been shown to play a pivotal role in post-translational regulation of gene expression via modulation of localization, stability and translation efficiency (Bashirullah A, Cooperstock R, Lipshitz H (2001) Spatial and temporal control of RNA stability. PNAS 98: 7025-7028). As noted above, mRNA delivery is desirable due to its non-transgenic nature; however, mRNA is a very fragile molecule, which is susceptible to degradation during the plant transformation process. Utilization of UTRs in plant mRNA transformations allow for increased stability and localization of mRNA molecules, granting increased transformation efficiency for non-transgenic genome modification.

Additionally, biolistic particle delivery systems may be used to transform plant tissue. Standard PEG and/or electroporation methods can be used for protoplast transformation. After transformation, plant tissue/cells are cultured to enable cell division, differentiation and regeneration. DNA from individual events can be isolated and screened for mutation. Any type of sequence-specific nuclease may be used to perform the methods provided herein as long as it has similar capabilities to TAL-effector nucleases. Therefore, it must be capable of inducing a double stranded DNA break at one or more targeted genetic loci, resulting in one or more targeted mutations at that locus or loci where mutation occurs through erroneous repair of the break by NH EJ or other mechanism (Certo M T, Gwiazda K S, Kuhar R, Sather B, Curinga G, et al. (2012) Coupling endonucleases with DNA end-processing enzymes to drive gene disruption. Nature methods 9:973-975. Christou, P. (1997) Rice transformation: bombardment. Plant Mol Biol. 35 (1-2):197-203). Such sequence-specific nucleases include, but are not limited to, ZFNs, homing endonucleases such as I-Scel and I-Crel, restriction endonucleases and other homing endonucleases or TALEN™s. In a specific embodiment, the endonuclease to be used comprises a CRISPR-associated Cas protein, such as Cas9 (Gasiunas, G., Barrangou, R., Horvath, P., Siksnys, V. (2012) Cas9-crRNA ribonucleoprotein complex mediates specific DNA cleavage for adaptive immunity in bacteria. PNAS 109(39):E2579-86).

Also in accordance with the invention there may be at least one polynucleotide comprising from 5′ to 3′, the expression regulatory element active specifically in plant vascular sheath cells, a nucleotide sequence which encodes a PHYB, active variant, or functional fragment thereof, and a terminator; and then a further polynucleotide encoding a genome editing nuclease, and optionally the same or further polynucleotide encoding a gRNA or crRNA which directs the genome editing nuclease protein to a desired locus in the genome of the plant, such that an exogenous PHYB, active variant, or functional fragment thereof under control of the vascular sheath regulatory element is inserted into the desired locus in the plant genome.

In some embodiments of the invention, there may be at least at least one polynucleotide comprises from 5′ to 3′, the expression regulatory element active specifically in plant vascular sheath cells, a nucleotide sequence which encodes a PHYB, active variant, or functional fragment thereof, such that the exogenous PHYB, active variant, or functional fragment thereof is inserted into the genome of the plant.

In some embodiments, methods may be used which do not employ induction of double strand DNA breaks to incorporate desirable DNA sequences. For example, prime editing is such a method that can be used to overwrite native nucleotide sequences. As will be familiar to a person of skill in the art, prime editing uses a DNA nickase enzyme coupled with an engineered reverse transcriptase enzyme to target and overwrite specific genomic regions with any DNA sequence. (See, for example, Kantor, A. et al., (2020) Int. J. Mol. Sci. 21: 6240 which provides a review of CRISPR-Cas9 DNA base editing and prime editing.) Prime-editors use an engineered reverse transcriptase fused to a nickase, such as a Cas9 nickase, and a prime-editing guide RNA (pegRNA). The pegRNA contains the sequence complimentary to the target sites that directs the nickase to its target sequence as well as an additional sequence spelling the desired sequence changes. Prime-editors may expand the scope of DNA editing to not all transition and transversion mutations, as well as small insertion and deletion mutations. Examples of nickases that may be employed in prime-editing include, but are not limited to, Cas9 nickases or Cas12 nickases. For example, a Cas 9 D10A Nickase or a Cas9 H840A Nickase may be employed. Further, a Cas9n can be employed using a paired nickase system with two different gRNA to extend the number of specifically recognized bases for target cleavage, which can improve specificity and help mitigate off-target phenomena. (See, for example, Khatodia, S., et al (2016) Front. Plant Sci. vol 7 page 506 which is another review article providing information about CRIPSR/Cas genome editing tools.)

Prime editing may be used to overwrite an endogenous native gene sequence, e.g. the expression regulatory element(s) of one copy of a native PHYB so that the resultant modified plants express PHYB specifically in at least some vascular sheath cells. Alternatively, prime editing could be used to further modify a native or exogenous sequence already introduced into the plant genetic material, e.g. by making a modification to the coding sequence of PHYB, e.g., so that it becomes an active variant such as YHB.

In some embodiments, the methods may employ a Cas endonuclease, wherein the Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. In some cases, the modified form of the Cas polypeptide has no substantial nuclease activity and is referred to as catalytically “inactivated Cas” or “deactivated Cas (dCas).” An inactivated Cas/deactivated Cas includes, for example, a deactivated Lapis Cas endonuclease (Lapis dCas). For example, in some embodiments, nuclease-deactivated Cas9 (dCas9) is used to implement such insertions. dCas proteins coupled with base editing enzymes (cytidine or adenine deaminases) can be used to modify RNA or DNA. In some embodiments, a direct effector fusion design may be employed, regulation (CRISPRi) or activation (CRISPRa) of targeted genes may be achieved by genetically fusing effector proteins—or their active domains—to dCas9 and expressing them as a single recombinant protein. For example, transcription activator domains (VP64, p65) or repressor domains (KRB, SID) may be fused to dCas9 to specifically increase or decrease target gene expression. In some embodiments, the effector domain(s) is recruited via functional scaffolds incorporated in the sgRNA-dCas9 complex, either via fusion to dCas9 or via RNA aptamers in a scaffolding RNA (scRNA). In other embodiments, Spatiotemporal control of effector activity is obtained via controlled recruitment of effectors to the sgRNA-dCas9 complex or the reconstitution of split-dCas9 directly fused to effectors via light- or chemical-inducible heterodimerization partners.

In other embodiments, methods of the invention may include the possibility of base editing which allows the modification of individual nucleotides. Base editing may employ DNA base editors, of which two classes have bene described: cytosine base-editors and adenine base-editors. DNA base-editors encompass two key components: a Cas enzyme for programmable DNA binding and a single-stranded DNA modifying enzyme for targeted nucleotide alteration. Where cytosine base-editors are used, cytosine deamination generates uracil, which base pairs as thymidine in DNA. Fusion of uracil DNA glycosylase inhibitor (UGI) inhibits the activity of uracil N-glycosylate (UNG), which may increase the editing efficiency of cytosine base-editing in cells. Where adenine base-editors are, adenosine deamination generates inosine, which has the same base pairing preferences as a guanosine in DNA. Collectively, cytosine and adenine base-editing can install all four transition mutations (C→J, T→C, A→G, and G→A). Thus, for example, the site directed action of a cytosine deaminase enzyme can be used to catalyse the conversion of a targeted cytosine base to uracil, which is then read as a thymine by native polymerases. Hence, there are multiple available options for both introducing vascular sheath expression regulation sequences to act on native phytochrome sequences, and for converting native PHYB to YHB sequences, as may be desired. The present invention also provides an isolated DNA polynucleotide comprising from 5′ to 3′, an expression regulatory element, e.g. a promoter, active specifically in a C₃ plant vascular sheath cell, a nucleotide sequence which encodes a PHYB, active variant, or functional fragment thereof, and a terminator.

In an embodiment of the invention, the promoter is a plant vascular sheath cell specific promoter which may be a bundle sheath cell specific promoter, or a mestome sheath cell specific promoter, or a promoter that is active specifically in both bundle sheath cells and mestome sheath cells.

In some embodiments, the isolated DNA polynucleotide may further comprise a nucleotide sequence which encodes a transcription factor, and a nucleotide sequence which encodes a second promoter (which is not a vascular sheath promoter described above) and which is recognized by the transcription factor, wherein the nucleotide sequence of the second promoter is upstream of the nucleotide sequence encoding a PHYB, active variant, or functional fragment thereof, and wherein the vascular sheath specific promoter drives the expression of the transcription factor.

The DNA polynucleotide may be synthesized in whole or in part; or optionally cloned in whole or in part. The promoter active specifically in C₃ plant vascular sheath cells, whether bundle sheath cells or mestome sheath cells (or both), may also be active in other cells of the vascular bundle, non-limiting examples of which include the phloem and/or xylem cells. The term “vascular bundle” as used in this application refers to all cells of the vascular bundle including the vascular sheath cells. The promoter active in vascular sheath cells may be also active in other non-vascular cell types, non-limiting examples of which include root cells, epidermal cells, or cells of the stomata such as guard cells. The promoter active in vascular sheath cells may also be active in extensions of the vascular sheath such as bundle sheath extension and the paraveinal mesophyll.

Also within the scope of the invention are promoters active specifically in C₃ vascular sheath cells, that is to say, these promoters are active in C₃ vascular sheath cells but not active in any other leaf tissue or leaf cell, but may be active in any of a number of possible plant cells or tissue types other than those found in leaves.

Terminator sequences are well known to a person of skill in the art and any appropriate terminator may be selected and used, e.g. as in the examples of the present invention wherein the terminator is Nosₜₑᵣ

Preferably, in any embodiment of the invention herein defined, the promoter is a vascular sheath promoter (e.g. a bundle sheath cell promoter, or a mestome sheath cell promoter, or a promoter that is expressed in both the bundle sheath and the mestome sheath). This can be a synthetic promoter comprised of various selected elements. For example, such a synthetic promoter may comprise a vascular sheath cell-specific transcription factor binding element upstream of the promoter element. There may be two or more transcription factor binding elements which may be the same or different. A plurality of such transcription factor binding elements may serve to enhance the activity and/or specificity of the promoter in vascular sheath cells.

For example, the promoter referred to above and comprised in the synthetic vascular sheath promoter may be selected from a minimal ZmUbi1 promoter, a NOS core promoter, a CHSA core promoter, or a minimal 35S promoter. Other minimal and/or core promoters can be used which are well known to a person of skill in the art. A preferred promoter has a nucleotide sequence of SEQ ID NO: 7 or SEQ ID NO: 10 or SEQ ID NO: 13 or a sequence of at least 80% identity therewith.

In other embodiments, the vascular sheath specific promoter may be derived from a gene that is expressed preferentially or specifically in the bundle sheath or mestome sheath (or both) of plants and so such a promoter is a naturally occurring promoter. The gene may be expressed in other cell types as well as vascular sheath cells, but preferably not expressed or very low expression in leaf mesophyll cells. The gene might be expressed also in guard cells, vascular sheath extensions, epidermal cells, guard cells, or other vascular tissues such as xylem and/or phloem; or elsewhere in the plant not being leaf tissue, e.g. flowers, fruits, roots, stems. Preferably such a naturally occurring vascular sheath promoter may be associated with a gene specifically expressed in plant bundle sheath cells or mestome sheath cells or both e.g. expressed only in bundle sheath cells and not expressed in any other plant tissue or cell type.

A vascular sheath specific promoter may be one from, for example, one of the following genes: Arabidopsis thaliana MYB76, Flaveria trinervia GLDP, Arabidopsis thaliana SULTR2;2, Arabidopsis thaliana SCR, Arabidopsis thaliana SCL23, Urochloa panicoides, PCK1, Zoysia japonica PCK, and Hordeum vulgare PHT1;1., including homologs of these genes. Although the promoters are designated by reference to a species of plant, of course the same or similar promoters may be found and used from different plant species of origin.

In some embodiments a vascular sheath promoter may be derived from non-plant organisms, such as the rice tungro bacilliform virus (RTBV) promoter.

The vascular sheath promoter may be derived from forward screens of mutant populations to identify promoters that drive gene expression in the vasculature.

In some embodiments vascular sheath preferential expression may be achieved by use of UTR sequences that when fused to the target coding sequence for PHYB, active variant, or functional fragment thereof confer cell specific expression of the protein even if transcript expression is driven by a constitutive promoter. Examples of such vascular sheath specific UTR elements include the UTR sequences from rubisco small subunit from either Flaveria bidentis (Patel et al. 2006. J Biol Chem 281(35):25485-91) or Amaranthus hypochondriacus (Patel et al. 2004. Plant Physiology 136(3): 3550-3561) both of which confer translational enhancement and preferential bundle sheath cell expression.

The PHYB or amino acid sequence variant which may be encoded in the DNA polynucleotides of the invention may correspond to any of the amino acid sequences of SEQ ID NO: 4, SEQ ID NO: 5, SEQ ID NO: 6, SEQ ID NO: 9, SEQ ID NO: 12. In addition to the aforementioned reference sequences, any of the coding sequences of the sequences identified by the accession numbers listed in Table 1 may instead be used as a reference sequence or sequences. In terms of variants of a reference sequence for PHYB, these may include sequences of at least 65% identity thereto; preferably at least 70% identity thereto; more preferably at least 80% identity thereto.

In exemplification of the invention a PHYB variant YHB SEQ ID NO: 4 is used and which is encoded by a nucleotide sequence of SEQ ID NO: 1. In further exemplification of the invention a PHYB variant YHB SEQ ID NO: 12 is used and which is encoded by a nucleotide sequence of SEQ ID NO: 11.

Therefore in polynucleotides of the invention, the nucleotide sequence encoding PHYB is any of SEQ ID NO: 1, SEQ ID NO: 2, SEQ ID NO:3, or SEQ ID NO:8, or SEQ ID NO: 11, or a sequence of at least 65% identity with any of said sequences; preferably a sequence of at least 70% identity with any of said sequences; more preferably a sequence of at least 80% identity with any of said sequences.

In certain embodiments of the invention, functional fragments of PHYB or variants thereof are employed. Such functional fragments have wild type phytochrome signalling activity, but lack light sensitivity. In other words, PHYB variants that are less than full length amino acid sequences and which are light insensitive as a result of the absence of the light sensing domains, or of essential amino acids for the light sensing function. Preferably the phytochrome fragments referred to herein consist of just the PAS and GAF domains.

The invention includes a DNA polynucleotide wherein the PHYB protein molecule, active variant, or functional fragment thereof encoded thereby is a light insensitive sequence variant; in other words there is substitution, deletion or insertion of one or more amino acids, resulting in light insensitivity of the protein whilst retaining the usual PHYB signalling activity function. The number of contiguous amino acid changes in such variants may be any number of amino acids selected from 1, 2, 3, 4, 5, 6, 7, 8, 9, 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39 or 40 amino acids. The number of amino acid changes which may have some but not wholly contiguous character may be selected from 1, 2, 3, 4, 5, 6, 7, 8, 9, 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39 or 40 amino acids.

In some embodiments, the present invention may comprise plasmids comprising a DNA polynucleotide as hereinbefore described, an origin of replication and a T-DNA right border repeat of a Ti or Ri plasmid, and at least one bacterial selectable marker. More often the plasmid also comprises a left border repeat of a Ti or Ri plasmid.

Plasmids in accordance with the invention may further comprise one or more other elements selected from: an enhancer, a plant selectable marker, a multicloning site, or a recombination site.

The invention also provides a Ti or Ri plasmid comprising a DNA polynucleotide as hereinbefore defined. The structure, modification, propagation and generation of vectors incorporating such plasmids is well known to a person of skill in the art.

In some embodiments, the invention may include a composition transformation of plant cells using a biolistic method. The composition therefore comprises microparticles coated with a DNA polynucleotide or a plasmid as hereinbefore defined. The microparticles may be of a metal or synthetic material. For example, microparticles may comprise tungsten or gold.

The invention also provides a bacterium comprising a plasmid as hereinbefore defined, i.e. a shuttle vector, and in some embodiments of this invention the bacterium is E coli.

Where a Ti or Ri plasmid is used to transform plant material this can be comprised in a suitable bacterium such as Agrobacterium sp.; preferably A. tumefaciens.

The invention includes any plants or plant materials, that is to say cells, tissues, organs, parts, seeds, or fruit, obtained or obtainable from any of the methods of the invention herein defined.

Products in accordance with the invention include plants which carry out C₃ photosynthesis in at least a part thereof, and which plants comprise a DNA polynucleotide as hereinbefore defined stably integrated into the genome thereof, and expressing PHYB, or active variants, or functional fragments as hereinbefore defined, in at least some of the vascular sheath cells (i.e. bundle sheath cells and/or mestome sheath cells). As already explained, this DNA polynucleotide may be introduced into plant genomes either by integrating a full-length promoter and PHYB, active variant, or functional fragment through genetic modification methods, or by gene editing the expression regulatory regions of native PHYB genes to alter their expression domains. Both approaches result in the same outcome i.e. the heritable expression of PHYB in vascular sheath cells. The PHYB gene, active variant, or functional fragment thereof, may be expressed in substantially all bundle sheath cells and/or mestome sheath cells.

The invention further includes a plant which carries out C₃ photosynthesis in at least a part thereof, wherein the plant has at least one copy of a PHYB gene, active variant, or functional fragment thereof as hereinbefore defined, and wherein the plant is genetically modified compared to an equivalent unmodified plant, wherein expression control element(s) of at least one copy of a PHYB gene, or active variant, or functional fragment thereof are modified to result in expression in at least some of the bundle sheath cells and/or the mestome sheath cells of the plant. In such plants, the expression control element is preferably a promoter which is active specifically in C₃ plant vascular sheath cells, as hereinbefore defined.

The coding sequence of the at least one PHYB gene may be the same as the native PHYB gene or genes in the plant. Therefore at least one native copy of the PHYB gene is modified to express in at least some of the vascular sheath cells of the plant. Consequently, in species with more than one copy of PHYB, at least one native PHYB gene remains under unmodified, native expression control.

In certain embodiments of modified plants, at least one PHYB gene is different to the other PHYB gene or genes in the plant.

Plants in accordance with the invention may be monocotyledons (monocots) or eudicotyledons (eudicots, dicots); preferably crop plants, e.g. fruits, vegetables, cereals, oilseed crops, legumes, biofuel crops, fibre crops, as are commonly used for food, animal feed, biofuel, or biomass production; also horticultural plants.

In preferred plants the DNA polynucleotide as defined herein is stably and heritably integrated into the genome thereof.

In some embodiments, the plants of the invention the PHYB gene expressed in at least some of the vascular sheath cells has an amino acid sequence of any of SEQ ID NO: 4, SEQ ID NO: 5, SEQ ID NO: 6, SEQ ID NO: 9, SEQ ID NO:12, or any of the sequence accessions listed in Table 1, or active variants, or functional fragments thereof as defined by encoding an amino acid sequence of at least 65% identity with any of said sequences; preferably a sequence of at least 70% identity with any of said sequences; more preferably a sequence of at least 80% identity with any of said sequences. In some embodiments, the PHYB gene has an amino acid sequence of SEQ ID NO: 4, SEQ ID NO: 5, SEQ ID NO: 6, SEQ ID NO: 9, SEQ ID NO:12, or any of the sequence accessions listed in Table 1. In other embodiments, the PHYB gene encodes an amino acid sequence that has at least 65% identity with any of SEQ ID NO: 4, SEQ ID NO: 5, SEQ ID NO: 6, SEQ ID NO: 9, SEQ ID NO:12, or any of the sequence accessions listed in Table 1. In other embodiments, the PHYB gene encodes an amino acid sequence that has at least 70% identity to SEQ ID NO: 4, SEQ ID NO: 5, SEQ ID NO: 6, SEQ ID NO: 9, SEQ ID NO:12, or any of the sequence accessions listed in Table 1. In other embodiments, the PHYB gene encodes an amino acid sequence that has at least 80% identity to SEQ ID NO: 4, SEQ ID NO: 5, SEQ ID NO: 6, SEQ ID NO: 9, SEQ ID NO:12, or any of the sequence accessions listed in Table 1. In other embodiments, the PHYB gene encodes an amino acid sequence that has at least 90% identity to SEQ ID NO: 4, SEQ ID NO: 5, SEQ ID NO: 6, SEQ ID NO: 9, SEQ ID NO:12, or any of the sequence accessions listed in Table 1, In plants of the invention where functional fragments of PHYB are expressed, these functional fragments are as hereinbefore defined.

The PHYB gene, active variant, or functional fragment thereof may be a light insensitive sequence variant, for example by way of one or more mutations involving substitution, insertion or deletion of amino acid residues. The PHYB gene, active variant, or functional fragment thereof may also be altered through substitution, insertion, or deletion of nucleic acid residues. In some embodiments, such as is described below, the PHYB sequence is that of the active variant YHB, encoding an amino acid sequence of SEQ ID NO: 4 or SEQ ID NO: 12 or a sequence of at least 65% identity therewith.

Plants in accordance with the invention may have chloroplasts present in vascular sheath cells such as bundle sheath cells and/or mestome sheath cells which may be larger than chloroplasts in equivalent cells of control unmodified plants grown under the same conditions for the same period of time.

Plants in accordance with the invention may have a photosynthetic rate greater than a control unmodified plant grown under the same conditions.

Plants in accordance with the invention may have a water use efficiency greater than in a control unmodified plant grown under the same conditions.

Plants in accordance with the invention may have enhanced photosynthetic efficiency compared to a control plant grown under the same conditions.

Plants in accordance with the invention may have enhanced photosynthesis which results in one or more of the following traits: enhanced growth rate, reduced time to flowering, faster maturation, enhanced seed yield, enhanced biomass, increased plant height, and increased leaf canopy area, when compared to a control plant grown under the same conditions.

The invention also provides a plant part, plant tissue, plant organ, plant cell, plant protoplast, embryo, callus culture, pollen grain or seed, derived or obtained from any kind of plant as described herein.

The invention also includes any processed plant product obtained from any plant described herein, wherein the processed product comprises a detectable nucleic acid sequence encoding (i) a PHYB gene or active variant or functional fragment thereof linked to a gene expression regulatory element active in at least some of the vascular sheath cells of a plant, or (ii) at least a portion of a polynucleotide of the invention. Such detection may employ techniques well known in the art such as PCR, qPCR, or application of any DNA or RNA sequencing technology of a suitably prepared sample of the processed plant material.

In summary from the above, the inventors have made a novel modification of C₃ plants which enhances photosynthetic capacity of the C₃ plant. In using the term “C₃” plant, this also includes plants which conduct C₃ photosynthesis in any part of the plant during any point of the plant life cycle (non-limiting examples include leaf sheath tissue, cotyledons, or photosynthetically active parts of the roots, stem and seed).

Previous attempts to boost plant productivity by increasing phytochrome signalling have either reduced photosynthesis and yield, or have achieved photosynthetic enhancement but only proportionately to chlorophyll investment (requiring more nitrogen investment) and resulted in reductions in water use efficiency and/or yield. These applications of this gene have also repeatedly produced undesirable side effects in crops: including dwarfing, canopy restructuring, delayed flowering, smaller tubers, and thicker leaves.

The overall effect of this C₃ plant modification is to boost photosynthesis, plant growth, and yield without any adverse effects on plant morphology, development or other agronomic traits. The invention is widely applicable to all C₃ plants and can generate 30% or higher increases in photosynthetic rate, growth rate, and seed yield, without any perturbation to normal plant development.

Overall the present invention achieves enhanced photosynthesis, growth, and yield with no observable negative or deleterious anatomical, physiological, biochemical or developmental effects on the modified plants.

### BRIEF DESCRIPTION OF THE DRAWINGS

Embodiments of the invention are further described hereinafter with reference to the Examples and accompanying Drawings, in which:

FIG. 1 depicts a simplified PHYB signalling cascade which contains non-limiting examples of genes which are affected, both at a transcript and/or at a protein level, by the activity of PHYB. PHYB activity releases several genes from repression, which then promote the development of photosynthetic capacity. Full gene names: PHYB=Phytochrome B, PIFs=Phytochrome Interacting Factors, COP1=Constitutive Photomorphogenic 1, GLK=Golden-2 Like Transcription factor, CGA1=Cytokinin Responsive GATA Factor 1, GNC=GATA, Nitrate-inducible, Carbon Metabolism-involved, HY5=Elongated Hypocotyl 5, HYH=HY5-Homolog.

FIG. 2 depicts a phylogenetic tree for Phytochrome B containing non-limiting examples of flowering plant members of the phytochrome B gene family. The tree is rooted at the base of the flowering plants. Representative species span the Monocots (Oryza sativa), and two major Dicot clades, the Rosids (Arabidopsis thaliana and Glycine max) and Asterids (Solanum lycopersicum). In the case of all three representative Dicot species, independent duplication of PHYB has resulting in the presence of two homologs of PHYB in each genome.

FIG. 3 shows a schematic of the genetic vector used to express the YHB protein in the vascular bundles of Arabidopsis thaliana by Agrobacterium mediated floral dip.

FIG. 4 shows the magnitude of YHB expression relative to a control gene eIF-4E1 in modified plants and a control unmodified plants. The control bar corresponds to wild type plants and the bar labelled “C12” corresponds to Arabidopsis plants containing the genetic vector for bundle sheath expression of YHB. Error bars indicate 95% confidence interval of the mean.

FIG. 5 shows the results of leaf thickness measurements for transgenic Arabidopsis plants containing the genetic vector for bundle sheath expression of YHB (bar labelled “C12”) and control Arabidopsis plants (bar labelled “control”). 95% confidence intervals are shown. ‘n.s.’ indicates that there was no significant difference between the C12 and control plants using a t-test. Comparisons are shown between control wildtype plants and one mutant line, however all 3 mutant lines investigated were consistent in their phenotypes.

FIG. 6 shows photosynthetic capacity as measured in the form of A/Ci curves, in transgenic Arabidopsis plants containing the genetic vector for bundle sheath expression of YHB (circles) and control plants (triangles).

FIG. 7 shows stomatal conductance measurement results for transgenic Arabidopsis plants containing the genetic vector for bundle sheath expression of YHB (circles) and control Arabidopsis plants (triangles).

FIG. 8 shows how there is enhanced water use efficiency when photosynthesis is operating maximally in transgenic Arabidopsis plants containing the genetic vector for bundle sheath expression of YHB (bar labelled “C12”) and control Arabidopsis plants (bar labelled “control”). 95% confidence intervals are shown. Asterisks indicate statistically significant differences at p<0.05 using a t-test. Comparisons are shown between control wildtype plants and one mutant line, however all 3 mutant lines investigated were consistent in their phenotypes.

FIG. 9 shows how there are larger chloroplasts in bundle sheath cells (BSC) but not mesophyll cells (MSC) of transgenic Arabidopsis plants containing the genetic vector for bundle sheath expression of YHB (bars labelled “C12”) when compared to control Arabidopsis plants (bars labelled “control”). 95% confidence intervals are shown. Asterisks indicate statistically significant differences at p<0.05 using a t-test, otherwise ‘n.s.’ indicates that there was no significant difference between the compared values. Comparisons are shown between control wildtype plants and one mutant line, however all 3 mutant lines investigated were consistent in their phenotypes.

FIG. 10 shows a comparison between chloroplasts of bundle sheath cells between control plants and transgenic Arabidopsis plants containing the genetic vector for bundle sheath expression of YHB. (A) is a representative image of bundle sheath cell chloroplasts of control plants. (B) is a representative image of bundle sheath cell chloroplasts of transgenic Arabidopsis plants containing the genetic vector for bundle sheath expression of YHB. (C) is a representative image of a bundle sheath cell chloroplast and a mesophyll cell chloroplast in a control plant. (D) is a representative image of a bundle sheath cell chloroplast and a mesophyll cell chloroplast in transgenic Arabidopsis plants containing the genetic vector for bundle sheath expression of YHB. BSC=bundle sheath cell, MSC=mesophyll cell, scale bar=2 microns.

FIG. 11 shows the results of stable carbon isotope measurements from leaf material. This data is consistent with increased refixation of respired carbon dioxide in transgenic Arabidopsis plants containing the genetic vector for bundle sheath expression of YHB (bar labelled “012”) when compared to control Arabidopsis plants (bar labelled “control”). 95% confidence intervals are shown. Asterisks indicate statistically significant differences at p<0.05 using a t-test. Comparisons are shown between control wildtype plants and one mutant line, however all 3 mutant lines investigated were consistent in their phenotypes.

FIG. 12 shows the results of vegetative growth rate measurements between weeks 2 and 3 after germination in transgenic Arabidopsis plants containing the genetic vector for bundle sheath expression of YHB (bar labelled “C12”) and in control Arabidopsis plants (bar labelled “control”). 95% confidence intervals are shown. Asterisks indicate statistically significant differences at p<0.05 using a t-test, otherwise ‘n.s.’ indicates that there was no significant difference between the compared values. Comparisons are shown between control wildtype plants and one mutant line, however all 3 mutant lines investigated were consistent in their phenotypes.

FIG. 13 shows the results of bolt height measurements, whereby taller bolts occur 35 days after germination in transgenic Arabidopsis plants containing the genetic vector for bundle sheath expression of YHB (bar labelled “C12”) when compared to control Arabidopsis plants (bar labelled “control”). 95% confidence intervals are shown. Asterisks indicate statistically significant differences at p<0.05 using a t-test. Comparisons are shown between control wildtype plants and one mutant line, however all 3 mutant lines investigated were consistent in their phenotypes.

FIG. 14 is a photograph of trays of transgenic and wildtype Arabidopsis plants containing the genetic vector for bundle sheath expression of YHB (“C12”) and control Arabidopsis plants (“wildtype”) undergoing normal photomorphogenesis.

FIG. 15 shows the results of measurement of time to bolting in transgenic Arabidopsis plants containing the genetic vector for bundle sheath expression of YHB (bar labelled “C12”) and control Arabidopsis plants (bar labelled “control”). 95% confidence intervals are shown. Asterisks indicate statistically significant differences at p<0.05 using a t-test. Comparisons are shown between control wildtype plants and one mutant line, however all 3 mutant lines investigated were consistent in their phenotypes.

FIG. 16 is a photograph showing silique production and above ground biomass at 8 weeks in transgenic Arabidopsis plants containing the genetic vector for bundle sheath expression of YHB (labelled C12) and control Arabidopsis plants (labelled wildtype). Comparisons are shown between control wildtype plants and one mutant line, however all 3 mutant lines investigated were consistent in their phenotypes.

FIG. 17 is a photograph of dry seeds collected from Arabidopsis plants containing the genetic vector for bundle sheath expression of YHB (in right hand tube), and also from control plants (left hand tube). Comparisons are shown between control wildtype plants and one mutant line, however all 3 mutant lines investigated were consistent in their phenotypes.

FIG. 18 shows measurements of dry seed biomass production in transgenic Arabidopsis plants containing the genetic vector for bundle sheath expression of YHB (bar labelled “C12”) and control Arabidopsis plants (bar labelled “control”). Measurements were taken at two different time points one “early” (seeds dried 6.5 weeks after germination) and one “late” (seeds dried 8 weeks after germination). 95% confidence intervals are shown. Asterisks indicate statistically significant differences at p<0.05 using a t-test. Comparisons are shown between control wildtype plants and one mutant line, however all 3 mutant lines investigated were consistent in their phenotypes.

FIG. 19 is a diagram of a proposed model for a novel, enhanced carbon refixation pathway in C₃ plants.

FIG. 20 shows ambient photosynthetic rate measured in ambient growth room conditions in transgenic wheat plants containing the genetic vector for bundle sheath expression of YHB (bar labelled “C12”) as compared to control wheat plants (bar labelled “control”). 95% confidence intervals are shown. Asterisks indicate statistically significant differences at p<0.05 using a t-test.

FIG. 21 is a photograph showing the enhancement in plant growth in a typical transgenic wheat plant containing the genetic vector for bundle sheath expression of YHB (right) as compared to a control wheat plant (left).

FIG. 22 shows the results of height measurements representing plant growth in transgenic wheat plants containing the genetic vector for bundle sheath expression of YHB (labelled “C12”) as compared to control wheat plants (labelled “control”) after seven weeks of growth. 95% confidence intervals are shown. Asterisks indicate statistically significant differences at p<0.05 using a t-test.

FIG. 23 shows the conservation of function among five vascular sheath promoters which have been shown to function in distantly related plant genera. Evolutionary relationship between 11 plant genera spanning three major plant clades (Rosids, Asterids and Monocots) are indicated by a phylogeny (branch lengths are arbitrary). For each of five promoters (SULTR2;2, GLDP, PCK, PHT1;1 and RBTV) arrows indicate the species of origin and arrowheads point to a distantly related species in which consistent vascular sheath expression has been demonstrated. Divergence time indicates how many millions of years it has been since the two species connected by arrows shared a common ancestor. For example, the Flaveria GLDP promoter drives consistent expression in Arabidopsis, despite both groups having diverged ˜125 million years ago.

FIG. 24 shows published experiments in which the function of PHYB orthologs from different species were shown to be conserved between distantly related plants. Phylogenies and evolutionary distance are depicted as in FIG. 23. Bold text indicates genera in which native PHYB expression has been altered, such as overexpression in Arabidopsis and Solanum (tomato) and gene knock out in Oryza (rice). Arrows indicate the origin of a PHYB gene, and point to plants in which this PHYB homolog has been overexpressed. For example, Arabidopsis PHYB was overexpressed in Arabidopsis, Solanum (tomato) and Miscanthus (silver grass). Regardless of PHYB origin species and recipient species, increased expression of PHYB results in consistent phenotypes (darker green leaves, shorter internodes and delayed flowering).

FIG. 25 shows conservation of functional domains in the amino acid sequences of a selection of PHYB proteins spanning>400 million years of land plant evolution. A cladogram indicates evolutionary relationships between Brassica napus, Solanum lycopersicum, Oryza sativa, Selaginella moellendorfii and Physcomitrella patens. In species that have duplicate copies of PHYB e.g. Brassica napus and Solanum lycopersicum, all copies of PHYB are shown. The characteristic PHYB domains are conserved in all PHYB proteins, and consist of domains (in order N to C terminus): PAS_2, GAF, PHY, PAS, PAS, HisKA, HATPase_c. Three key events in land plant evolution are annotated with crosses: the emergence of vascular plants (>400 million years ago (mya)), flowering plants (>160 mya) and Brassicaceae (>40 mya). Branch lengths are arbitrary and not reflective of evolutionary distance.

FIG. 26 shows the expression of three different Brassica napus PHYB genes (in Transcipts Per Million) in the leaves of 16 different cultivars.

FIG. 27 shows the alignment of the 50 base pairs flanking the single nucleotide that it is necessary to change in order to convert Brassica napus PHYB into a constitutively active form that is equivalent of Arabidopsis thaliana YHB (highlighted). Asterisks beneath the multiple sequence alignment indicate nucleotides that are conserved in all three full length Brassica napus copies of the PHYB gene. The 14 underlined bases show points of variation between the PHYB copies which allow individual copies to be targeted for editing.

FIG. 28 shows two designs which exemplify different approaches to gene editing PHYB expression in two species: The Solyc05g053410 tomato PHYB gene (top) and the soybean Glyma.09G035500 gene (bottom). PHYB genomic regions are depicted and annotated with native exons, 5′ and 3′ Untranslated Regions (UTRs), and inserted promoter and enhancer sequences which would confer vascular bundle expression in these genes. Genomic features are labelled according to their position relative to the start codon (starting at position 0).

### DETAILED DESCRIPTION

In the following passages, different aspects of the invention are explained in more detail. Each aspect explained or defined may be combined with any other aspect or aspects, unless explicitly indicated to the contrary. In particular, any feature indicated as being preferred or advantageous may be combined with any other feature or features indicated as being preferred or advantageous.

Conventional techniques of botany, microbiology, tissue culture, molecular biology, chemistry, biochemistry, recombinant DNA technology, and bioinformatics for use in employing the present invention are all readily known and available to a person of average skill in the art. Specific techniques are explained fully in the literature.

The inventors have generated a system comprised of a vascular sheath specific regulator of gene expression and a regulator of chloroplast activation that together increase photosynthesis and yield related traits. The inventors have demonstrated that this technology is broadly applicable to C₃ plants by showing that it works in both eudicots (for example Arabidopsis thaliana) and in monocotyledons (for example wheat). The inventors have shown that this technology works irrespective of the species origin of the PHYB gene and irrespective of the vascular sheath promoter that is used. A key aspect of this invention is that PHYB, active variants, or functional fragments thereof are expressed in vascular sheath cells (which may include other cells of the vasculature or vasculature sheath extensions as hereinbefore defined) and not leaf mesophyll cells. Transgenic plants containing this system surprisingly and advantageously do not display developmental defects associated with YHB or PHYB overexpression. The transgenic plants undergo normal photomorphogenesis (no dwarfing, reduced apical dominance, delayed flowering, or decreased water use efficiency), have the same leaf thickness as control plants, and flower normally. However, these plants have higher photosynthetic rates, grow faster, have enhanced water use efficiency, mature to flowering stage sooner, produce more fruiting structures and produce significantly more seeds. The effects are dramatic, with yield increases upward of 30% in greenhouse trials.

The inventors have achieved what has not hitherto been possible, which is a manipulation of PHYB expression in planta to improve each of photosynthesis, plant growth and yield without disrupting plant development. The unexpected finding of the inventors is that combined improvements are achievable separately from the disruptive aspects of PHYB expression by expressing PHYB additionally only in the vascular bundle or component cells thereof of plants.

The terms “peptide”, “polypeptide” and “protein” are used interchangeably herein and refer to amino acids in a polymeric form of any length, linked together by peptide bonds.

The terms “altered”, “changed” and “modified” may be used interchangeably herein. A control plant as used herein is a plant which has not been modified. Accordingly, the control plant has not been genetically modified to alter either expression of a polynucleotide of the invention as described herein. The control plant may be a wild type (WT) plant. Even if a plant were transgenic, but not in respect of the polynucleotide of the invention then it could function as a control plant. The WT or control need not be too specific, so long as it may provide a reliable reference against which the vascular bundle sheath expression of PHYB can be compared against in a modified plant material.

The terms “increase”, “improve” or “enhance” are used interchangeably herein.

The term “specific” as used herein may be considered equivalent to “exclusive” or strongly preferential.

### Vascular Sheath and Vascular Sheath Cells

In C₃ plants (most crops), the cells surrounding the leaf veins (i.e. the vascular sheath) are known as bundle sheath cells. In Dicotyledonous plants the bundle sheath is made up of a single layer of cells that encircle the vein, while in Monocotyledonous plants the bundle sheath can be made up of a single layer of cells or two concentric layers of cells (A. Fahn, Plant Anatomy Pergamon Press 1995). When there are two layers of cells the outer cell layer is commonly referred to as the bundle sheath and the inner cell layer is commonly referred to as the mestome sheath (A. Fahn, Plant Anatomy Pergamon press 1995). When two layers are present, both layers together make up the bundle sheath (A. Fahn, Plant Anatomy Pergamon press 1995). Thus, bundle sheath is a term used to describe either a single layer of bundle sheath cells, or a two-layer system comprised of an outer bundle sheath layer and an inner mestome sheath layer. As used throughout this specification, the terms “bundle sheath”, “bundle sheath cells”, “vascular sheath”, or “vascular sheath cells” may be used interchangeably, encompassing all types of bundle sheath cell layers unless the context clearly dictates otherwise. Bundle sheath cell layers (i.e. the single bundle sheath layer, or the outer bundle sheath and the inner mestome sheath) may contain chloroplasts. The number of chloroplasts in these bundle sheath layers may be the same or fewer than in mesophyll cells and in some cases bundle sheath cells may be devoid of chloroplasts. Furthermore, if they are present in Ca plants the size of the chloroplasts in bundle sheath cell layers is generally much smaller than in mesophyll cells (A. Fahn, Plant Anatomy: Pergamon Press (1995)). The bundle sheath cells encircle veins so they are ideally situated to ensure good water supply and for loading sugars into veins for distribution to growing plant structures.

### Bundle Sheath Specific Expression

The term “specific” when used in relation to gene expression describes the biological phenomenon of enhanced gene expression within a limited subset of cell types within a plant. The term “bundle sheath specific expression” is used synonymously with “vascular sheath specific expression” to describe the phenomenon whereby the gene being expressed is expressed to a substantially higher level in bundle sheath cells than in the surrounding mesophyll cells within the leaf. This does not preclude the gene from being expressed in other non-mesophyll cells within the leaf or within the plant, just that the level of expression in the bundle sheath is high and the level of expression in the leaf mesophyll is low. The gene may also be expressed in other vascular cell types in addition to the vascular sheath cells. These cell types include some or all of the cells of the vascular bundle such as xylem and/or phloem and associated cell types. The gene may also be expressed in non-vascular cells such as guard cells, vascular sheath extension cells, bundle sheath extension cells, epidermal cells, paraveinal mesophyll cells (which are an extension of the bundle sheath and not mesophyll cells); or elsewhere in the plant not being leaf tissue, e.g. flowers, fruits, roots, stems. The key determinant is that expression is activated in the bundle sheath and not the mesophyll.

### Phytochrome Proteins for Use in the Invention

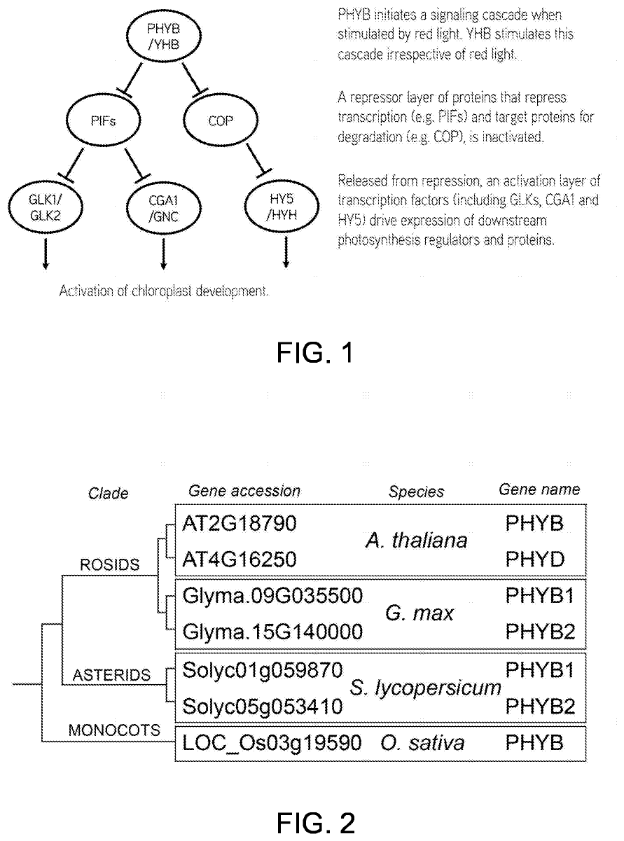

“PHYB” (Phytochrome B) as hereinbefore defined is a regulatory photoreceptor. As shown in FIG. 1, PHYB activity induces a regulatory cascade by inhibiting the action of transcriptional repressors, such as the Phytochrome-Interacting Factors (PIFs), and of proteins that target other proteins for degradation (such as Constitutive Photomorphogenic 1, COP1) (Legris et al., (2019) “Molecular mechanisms underlying phytochrome-controlled morphogenesis in plants.” Nat. Comms. 10:5219). In the dark, this layer of repressor proteins inhibit the transcription of photosynthesis proteins by preventing the accumulation of transcription factors that activate expression of photosynthesis proteins, such as Elongated Hypocotyl 5 (HY5 and its paralog HYH), Golden-2 Like transcription factors (GLK1 and its paralog GLK2), and Cytokinin Responsive GATA Factor 1 (CGA1 and its paralog GNC) (Wang et al., (2017) “Transcriptional control of photosynthetic capacity: conservation and divergence from Arabidopsis to rice.” New Phytol., 216: 32-45.). The transcription of hundreds of genes, including core machinery required to carry out photosynthesis, have been attributed to the action of these three groups of transcription factors. In the light, the PHYB proteins present in mesophyll cells are activated and release these transcription factors from repression. The resulting transcriptional cascade ultimately gives rise to chloroplast development and photosynthetic activation.

PHYB from any plant species may be used in embodiments of the invention, whether that PHYB protein, active variant or functional fragment thereof is expressed in the same plant (homologous expression) or in a different plant (heterologous expression).

The term “active variant” and/or “functional fragment” as used herein in relation to PHYB refers to a variant or fragment of a PHYB gene or peptide sequence which retains the signal activating function of PHYB. An active variant also comprises a variant of the gene of interest encoding a peptide which has sequence alterations that do not affect the signal activating function of the resulting protein, for example in non-conserved residues.

The invention also includes functional fragments of PHYB and any variants of a PHYB protein, for use in accordance with any aspect of the invention.

### Sequence Identities and Orthology

The term “variant” as used herein used in relation to a given PHYB protein from a plant species, or a functional fragment thereof, means any PHYB ortholog of differing amino acid sequence from other plant species. Such variants may be expressed in terms of a percentage identity to any of the reference nucleotide reference sequences disclosed herein (i.e. SEQ ID NO: 1, SEQ ID NO: 2, SEQ ID NO: 3 or SEQ ID NO: 8 or SEQ ID NO: 11). In terms of percentage identity to an amino acid reference sequence, such as SEQ ID NO:4, a variant of PHYB may have, in increasing order of preference, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to that amino acid reference sequence.

The following table provides a non-exhaustive list of accession numbers for PHYB orthologs in 50 commercially grown plant species. Orthologs of the Arabidopsis PHYB gene were found in the NCBI publicly available sequence database. More than one PHYB accession was found for many species, indicative that PHYB has duplicated in different plant lineages; many of these paralogues arose as result of whole genome duplications. For each species, one representative, full-length, orthologous amino acid sequence was compared to Arabidopsis and wheat PHYB orthologs (AT2G18790.1 and Traes_4 AS_1F3163292.1, respectively), and percentage identity to each was quantified using multiple sequence alignments generated by Clustal Omega 2.1 with default parameters (generating alignments with mBed-like clustering guide trees and hidden Markov models using HHalign). The median PHYB percentage identity of these orthologs relative to Arabidopsis or wheat PHYB orthologs was ˜75%. There were several examples where less than 75% identity was shared between a PHYB ortholog in a given species and both the Arabidopsis and wheat orthologs. For example, Daucus carota (carrot) and Solanum lycopersicum (tomato) PHYB orthologs were >70% identical to either the Arabidopsis or wheat PHYB orthologs. Likewise, PHYB orthologs in more distantly related gymnosperm species such as Picea abies and sitchensis (spruces) were just 66-68% identical to either Arabidopsis or wheat PHYB proteins at the amino acid level. Recently duplicated PHYB paralogues fall within the PHYB similarity range indicated by the table, but more distantly related phytochromes do not. For example, a multiple sequence alignment of Arabidopsis PHYB [SEQ ID NO: 5], PHYD [SEQ ID NO: 9] and PHYA (NCBI accession NP_001322907.1), amino acids indicated that while PHYB and paralogous PHYD share 81.98% identity, PHYA shares just 52.35% identity with PHYB, and 52.20% with PHYD.

| | | Arabidopsis | Wheat |
|---|---|---|---|
| Species name | Accession | identity % | identity % |
| Arachis hypogaea | XP_016197860.1, XP_025694495.1 | 76.95 | 75.00 |
| Beta vulgaris | XP_010671734.1, XP_010671735.1 | 76.40 | 72.94 |
| Brassica carinata | KAG2315801.1, KAG2294593.1, | 72.10 | 72.05 |
| | KAG2271748.1, KAG2306049.1 | | |
| Brassica napus | XP_013741043.1, XP_022555281.1, | 90.25 | 71.37 |
| | CAF2100974.1, XP_022575358.1, | | |
| | XP_022559055.1, CAF1933771.1 | | |
| Brassica oleracea | XP_013585553.1, XP_013628810.1 | 92.53 | 71.30 |
| Camelina Sativa | XP_010489599 | 94.93 | 71.84 |
| Camelina sinensis | THG18270.1, THG09607.1, KAF5941548.1, | 77.51 | 75.62 |
| | XP_028060883.1, KAF5950816.1, | | |
| | XP_028079860.1 | | |
| Cannabis sativa | XP_030506649.1, KAF4358918.1, | 76.21 | 75.35 |
| | XP_030506648.1 | | |
| Capsicum annuum | XP_016581708.1, PHT94245.1 | 78.11 | 76.08 |
| Chenopodium | XP_021730340.1, XP_021774295.1 | 76.32 | 74.20 |
| quinoa | | | |
| Cicer arietinum | XP_004486544.1 | 75.09 | 74.26 |
| Citrus × sinensis | KDO71942.1 | 78.50 | 75.29 |
| Coffea arabica | XP_027120998.1, XP_027115886.1 | 77.53 | 75.53 |
| Corchorus | OMO53500.1 | 79.28 | 75.98 |
| capsularis | | | |
| Cucumis sativus | XP_004134246.2 | 78.48 | 75.91 |
| Cucurbita pepo | XP_023526818.1, XP_023540778.1, | 73.74 | 73.07 |
| | XP_023540779.1 | | |
| Daucus carota | KZM85596.1 | 71.67 | 71.54 |
| Elaeis guineensis | XP_010921452.1, XP_010938231.2 | 75.09 | 72.02 |
| Eucalyptus grandis | KCW87973.1 | 77.76 | 77.67 |
| Fragaria vesca | XP_004295077.1 | 77.44 | 76.19 |
| Glycine max | NP_001240097.1, XP_006597696.1 | 76.19 | 74.80 |
| Gossypium | XP_016700852.1, XP_016677281.1 | 78.81 | 73.99 |
| hirsutum | | 72.59 | 72.04 |
| Helianthus annuus | XP_022022035.1, XP_021987936.1 | | |
| Hevea brasiliensis | KAF2312734.1, XP_021668699.1 | 78.72 | 77.17 |
| Hordeum vulgare | KAE8810763.1 | 71.60 | 99.14 |
| Jatropha curcas | XP_012084068.1, XP_012084071.1, | 78.68 | 76.53 |
| Juglans regia | XP_018805735.2 | 78.69 | 76.04 |
| Lactuca sativa | XP_023763453.1 | 75.24 | 73.48 |
| Malus domestica | XP_008368332.2, RXH80138.1 | 76.30 | 74.46 |
| Manihot esculenta | XP_021607077.1 | 78.48 | 77.17 |
| Medicago sativa | ACU21557.1, ACU21558.1 | 75.22 | 73.05 |
| Musa acuminata | AOA13605.1 | 71.12 | 77.51 |
| Nicotiana tabacum | XP_016456908.1, XP_016458771.1, | 73.22 | 71.37 |
| | XP_016441820.1, ALN38804.1, P29130.2, | | |
| | XP_016454809.1 | | |
| Olea europaea | XP_022851738.1 | 77.36 | 75.13 |
| Oryza sativa | XP_015631282.1 | 74.82 | 93.14 |
| Picea abies | AJE63445.1 | 67.23 | 67.79 |
| Picea sitchensis | ACN40636.1 | 66.87 | 67.35 |
| Pisum sativum | AAF14344.1 | 75.16 | 73.01 |
| Phaseolus vulgaris | XP_007147366.1 | 76.02 | 75.66 |
| Populus | AAG25725.1 | 71.34 | 75.15 |
| trichocarpa | | 79.25 | 76.65 |
| Prunus persica | XP_007227356.1 | | |
| Ricinus communis | XP_002519230.1 | 77.98 | 76.16 |
| Sesamum indicum | XP_011100755.1, XP_011071377.1, | 75.53 | 74.17 |
| | XP_020555118.1, | | |
| Solanum | NP_001317100.1, NP_001293131.1 | 71.70 | 70.74 |
| lycopersicum | | | |
| Solanum | XP_006355734.1, XP_006358209.1 | 71.98 | 70.83 |
| tuberosum | | | |
| Spinacia oleracea | KNA10134.1, AAA17825.1, XP_021862546.1, | 75.29 | 73.23 |
| | KNA10706.1, XP_021858666.1 | | |
| Theobroma cacao | EOY06733.1 | 79.70 | 76.25 |
| Triticum aestivum | KAF7042404.1, KAF7054102.1, | 72.08 | 100.00 |
| | KAF7049239.1, AAX76779.1, KAF7054101.1 | | |
| | KAF7042406.1 | | |
| Vigna unguiculata | QCD77474.1, XP_027931104.1, QCE13780.1 | 76.88 | 76.84 |
| Vitis vinifera | CBI22877.3 | 78.19 | 77.09 |

The overall sequence identity may be determined using a global alignment algorithm known in the art, such as the Needleman Wunsch algorithm in the program GAP (GCG Wisconsin Package, Accelrys).

More examples of suitable PHYB genes can also be readily identified by a skilled person through ortholog finding programs such as OrthoFinder (Emms and Kelly. Genome Biology 2019. 20: 238). The function of such genes can be identified as described herein and a skilled person would thus be able to confirm the function when expressed in a plant.

FIG. 2 shows the PHYB gene family for four representative plant species spanning three major clades of flowering plants (Rosids, Asterids and Monocots). The tree is rooted at the origin of flowering plants and branch lengths are arbitrary. The phytochrome B gene duplicated in the lineage that gave rise to the Brassicaceae resulting in a paralogous gene pair that are known as Phytochrome B (AT2G18790) and Phytochrome D (AT4G16250) in Arabidopsis thaliana. Likewise, Glycine max (soybean) and Solanum lycopersicum (tomato) have two copies of PHYB, which arose from independent gene duplication events. In these species, these duplicates are instead called PHYB1 and PHYB2. Hence, O. sativa (rice) PHYB is equally related to both A. thaliana PHYBs (B and D) and to both S. lycopersicum PHYBs (1 and 2). In species with multiple copies of PHYB there is evidence that both copies function redundantly. For example, overexpression of either S. lycopersicum PHYB1 or PHYB2 in S. lycopersicum produces the same phenotype (Husaineid et al., (2007) “Overexpression of homologous phytochrome genes in tomato: exploring the limits in photoperception” J. Exp. Bot. 58: 615-626). Thus, as used in this application, the term PHYB comprises the complete PHYB gene family exemplified by the representative members of this gene family shown in FIG. 2, and includes all PHYB paralogs such as Phytochrome D.

Where the bundle sheath cell specific promoter is concerned, all variants and orthologs of these are included in the invention. Where there is a reference nucleotide sequence for such a promoter, then such variants and orthologs include nucleotide sequences of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to the reference promoter sequence.

The degree of sequence identity of any polynucleotides described in connection with the invention may, instead of being expressed as a percentage identity to reference sequence, may instead be defined in terms of hybridization to a polynucleotide of any of the reference sequences [SEQ ID NO: 1, SEQ ID NO: 2, SEQ ID NO: 3 or SEQ ID NO: 4 or SEQ ID NO: 8 or SEQ ID NO: 11] disclosed herein. Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

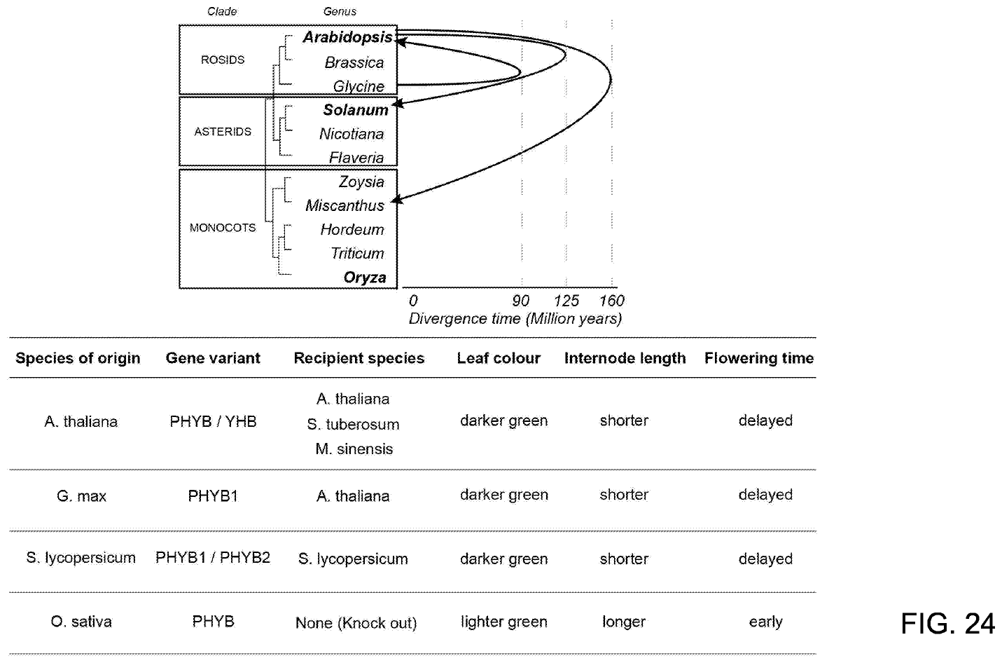

PHYB is a highly conserved protein and its functions are highly conserved throughout all vascular plants. This has repeatedly been demonstrated by either increasing expression of native PHYB proteins, expressing exogenous PHYB proteins from other plant species, or knocking out native PHYB genes. FIG. 24 summarises illustrative examples in which PHYB expression has been altered by genetic manipulation: Overexpressing either native PHYB or YHB in Arabidopsis (Su & Lagarias, (2007) Plant Cell. 19(7): 2124-2139), expressing Arabidopsis PHYB in Solanum (potato) (Thiele et al., (1999) Plant Physiology. 120: 73-81) and in Miscanthus (switchgrass) (Hwang et al., (2014) International Journal of Photoenergy), overexpressing native tomato PHYB genes in Solanum (either of the two PHYBs in the tomato genome, Husaineid et al., (2007) J. Exp. Bot. 58: 615-626.), Glycine (soy) PHYB in A. thaliana (Wu et al., (2011) PLoS ONE 6(11)), or knocking out phytochromes in Oryza (rice) (Takano et al., (2009) PNAS. 106(34): 14705-14710). Even though this wealth of experimental evidence contains diverse species and PHYB proteins (Miscanthus and Arabidopsis diverged ˜160 million years ago), distinctive phenotypic effects are consistently observed across these experiments: Consistent changes in chlorophyll (leaf colour), dwarfing (internode length) and flowering time are observed in all plant species, irrespective of the source species of the PHYB gene that is expressed. Thus, the PHYB gene from any plant species can provide the function of PHYB in any other plant species when expressed in that plant. Therefore, any PHYB protein can be expected to induce similar mechanistic functions when expressed in any vascular plant.

### Functional Fragments

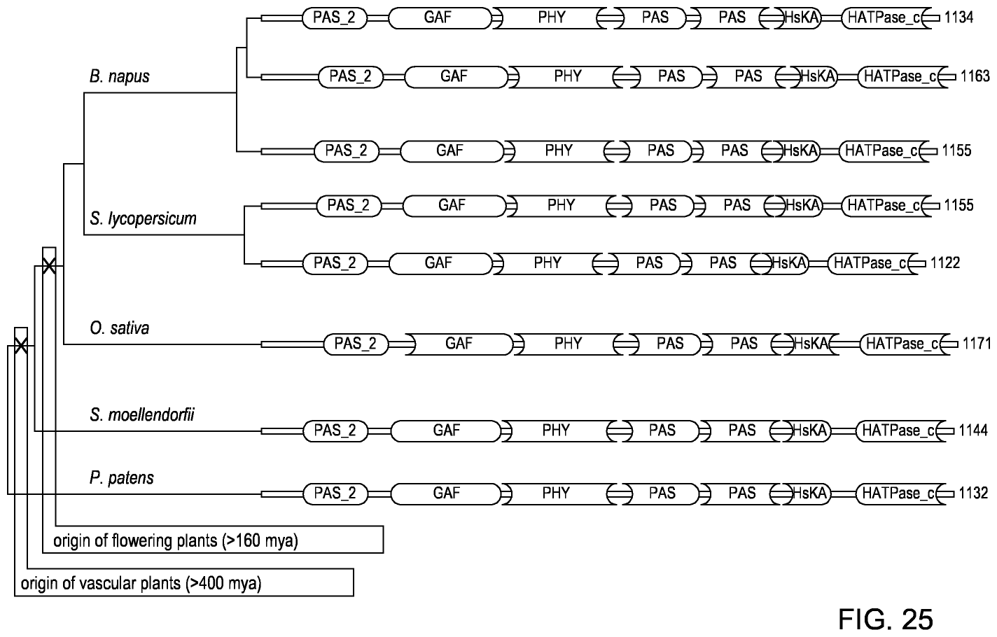

PHYB proteins are typically comprised of 7 easily recognisable protein domains. These comprise three Per-Arnt-Sim (PAS) domains (either PF08446 and/or PF00989), a GAF domain (PF01590), a PHY domain (PF00360), a His Kinase A phospho-acceptor domain (PF00512), and a GHKL domain (PF02518). FIG. 25 illustrates these characteristic PHYB functional domains from PHYB proteins found in five diverse land plant species, Brassica napus, Solanum lycopersicum, Oryza sativa, Selaginella moellendorfii and Physcomitrella patens. Despite spanning>400 million years of evolution (Physcomitrella to Brassica) and multiple instances of gene duplications (e.g. Brassica napus and Solanum lycopersicum), all PHYB proteins are of similar length and contain the same arrangement of PAS_2, GAF, PHY, PAS, HisKA and HATase_c(/GHKL) domains. Domains were identified using the EBI HMMR tool (Potter et al., (2018) Nucleic Acids Research 46:W200-W204).

Though these protein domains are highly conserved, truncated versions of the PHYB gene can also function to initiate PHYB signalling. For example, Oka et al. (2004) “Functional Analysis of a 450-Amino Acid N-Terminal Fragment of Phytochrome B in Arabidopsis” Plant Cell. 16(8): 2104-2116 showed that a 450-amino acid fragment of PHYB, which lacks the PHY domain (PF00360), the His Kinase A phospho-acceptor domain (PF00512), and the GHKL domain (PF02518), could initiate PHYB signal transduction when targeted to the nucleus. Thus, functional fragments of PHYB can provide PHYB signalling and such functional fragments are included in this invention.

Vascular Sheath (i.e. Vascular Bundle, Bundle Sheath and/or Mestome Sheath) Promoters

A person of skill in the art is well aware of many vascular bundle, vascular sheath, bundle sheath or mestome sheath specific promoters.

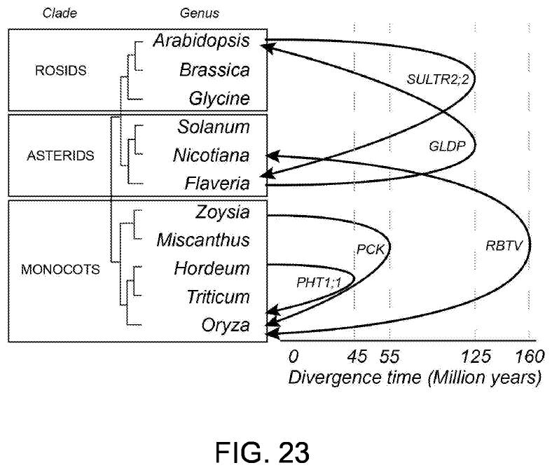

There are such promoters that have been isolated from several different species that a person of average skill in the art will expect to work across diverse plant species; five such examples are illustrated in FIG. 23. The promoter from the gene encoding the P-Subunit of Glycine Decarboxylase in Flaveria trinervia, described in Engelmann et al (2008) Plant Physiology 146(4):1773-1785, drives expression in bundle sheath cells and vascular bundles in Flaveria bidentis and also in the distantly related eudicot species Arabidopsis thaliana. These species last shared a common ancestor ˜125 million years ago, hence the activity of this promoter is conserved across eudicots (Zeng et al New Phytol. 2017 May; 214(3):1338-1354). Indeed, additional research on the GLDP promoter has revealed that its cross functionality between species is conferred by a regulatory sequence that is conserved across the Brassicaceae family, including Arabidopsis, Brassica, Capsella and Moricandia species (Adwy et al. The Plant Journal 2015 November; 84(6) and Adwy et al. Plant Gene 2019 June; 18). Similarly, the promoter for the gene encoding the sulphur transporter SULTR2;2 in Arabidopsis thaliana described in Kirschner et al (2018) Journal of Experimental Botany 69(20): 4897-4906, drives expression in the bundle sheath and veins of Arabidopsis and also in the distantly related species Flaveria bidentis. In yet further examples, the promoters from genes that are expressed in the bundle sheath cells of C₃ plants can also confer bundle sheath specific expression in those plants. This is illustrated by the promoter from the MYB76 gene from Arabidopsis thaliana which is expressed in the vascular bundles of Arabidopsis. The promoter from this gene is sufficient to drive vascular bundle specific expression of reporter genes in Arabidopsis, and was found in a highly conserved region of the genome among members of the Brassicaceae family (Kneřová, et al biorxiv https://doi.org/10.1101/380188), a trait it shares with the cross-functional GLDP promoter. There are numerous other such examples of promoters which, when fused to reporter genes, drive expression in vascular bundles. For example, the promoters from genes which when knocked out give reticulate phenotypes provide dominant (or exclusive) expression in vasculature or bundle sheath (BS) cells (Lundquist et al Molecular Plant. 2014 January; 7(1):14-29). Also, the promoters of both the SCARECROW (SCR) and SCARECROW-LIKE 23 (SCL23) genes drive expression of reporter genes specifically in bundle sheath cells (Cui et al. The Plant Journal. 2104 78(2): 319-327).

There are also bundle sheath cell promoters described in the literature for Monocots. For example, Nomura et al (2005) Plant Cell Physiology. 46(5): 754-61 which shows that Zoysia japonica PCK promoter works to drive expression in rice bundle sheath. Similarly, the Urochloa panicoides PCK1 promoter directs bundle sheath expression of reporter genes in rice and maize (Suzuki and Burnell. Plant Science. 2003 165(3):603-611). Also, Kloti et al (1999) Plant Molecular Biology 40(2): 249-266 shows rice tungro bacilliform virus promoter working in vascular bundles and other vascular cells. This promoter works in both Monocots (rice) and Dicots (tobacco) to drive expression in vascular bundles, despite these species having diverged ˜160 million years ago. Petruccelli et al 2001 PNAS 98(13) 7635-7640. Also, Schünmann et al (2004) Plant Physiol. 136(4): 4205-4214 which shows rice bundle sheath expression using the barley Pht1;1 promoter (see FIG. 31 therein). Since vascular bundle tissue is a universally conserved feature of vascular plant leaves, bundle sheath promoters from eudicots, e.g. those that have been published and found to work in distantly related species such as Asterids and brassicas, will also be expected by a person of average skill in the art to work in Monocots and vice versa (as in the case of the rice tungro bacilliform virus promoter described above that works in both Monocots and euicots). Moreover, there is a large diversity of bundle sheath promoters already known to a person of average skill in the art, and any of these promoters (either individually or in combination) would be suitable to drive the expression of PHYB or YHB in the vascular bundles or bundle sheath cells of any plant.

### Recombinant Constructs

Any suitable cloning system may be used. For example, Golden Gate modular cloning system described in Weber, E. et al (2011) PLoS ONE doi.org/10.1371/journal.pone.0016765. Otherwise genetic constructs can be fully synthesized de novo, or assembled using other molecular biology approaches.

PHYB, active variant or functional fragment sequences of the invention may be operably linked for transcription and expression, whether directly or indirectly to the vascular sheath promoter(s) employed in the invention.

### Plant Transformation

Transformation of plants is now a routine technique in many species. Advantageously, any of several transformation methods may be used to introduce the gene of interest into a plant. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, can also be produced via Agrobacterium tumefaciens mediated transformation. Such routine methods are also used to introduce genome editing proteins such as CRISPR Cas nucleases, base editors and other genome editing nucleases. Collectively or in isolation these genome editing nucleases can be used to edit native PHYB gene sequences to introduce vascular sheath promoter sequences, vascular sheath regulatory elements, or convert native PHYB sequences to active variants or functional fragments.

Transformation methods are well known in the art. Thus, according to the various aspects of the invention, a polynucleotide of the invention is introduced into a plant and expressed as a transgene. The nucleic acid sequence is introduced into said plant through a process called transformation. The term “introduction” or “transformation” is used to encompass “transformation”, “transfection”, “transduction” and all such methods that result in the transfer of an exogenous polynucleotide into a host plant cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host plant genome. The resulting transformed plant cell may then be used to regenerate a transformed plant in a manner well known in the art.

To select transformed plants, plant material obtained in the transformation is, as a rule, subjected to selective conditions so that transformed plants can be distinguished from untransformed plants. For example, seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, for instance using Southern analysis or whole genome sequencing, for the presence of the gene of interest, copy number and/or genomic organisation. Alternatively or additionally, expression levels of the newly introduced DNA may be monitored using Northern and/or Western analysis and/or RNA-Seq, each being well known in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Altered plants in accordance with the invention advantageously provide better yield characteristics. Yield characteristics, also known as yield traits may comprise one or more of the following non-limitative list of features: yield, biomass, seed yield, seed/grain size, starch content of grain, early vigour, greenness index, increased growth rate, increased water use efficiency, increased resource use efficiency. The term “yield” in general means a measurable produce of economic value, typically related to a specified crop, to an area, and to a period of time. Individual plant parts directly contribute to yield based on their number, size and/or weight, or the actual yield is the yield per square meter for a crop and growth period, which is determined by dividing total production (includes both harvested and appraised production) by planted square metres. The term “yield” of a plant may relate to vegetative biomass (root and/or shoot biomass), to reproductive organs, and/or to propagules (such as seeds and tubers) of that plant. Thus, according to the invention, yield comprises one or more of, and can be measured by assessing one or more of: increased seed yield per plant, increased seed filling rate, increased number of filled seeds, increased harvest index, increased viability/germination efficiency, increased number or size of seeds/capsules/pods, increased growth or increased branching, for example inflorescences with more branches, increased biomass, increased grain fill, increase tuber biomass. Preferably, increased yield comprises an increased number of grains/seeds/capsules/pods, increased biomass, increased growth, increased number of floral organs, increased floral branching or increased tubers. Yield is usually measured relative to a control plant.

Preferably, a plant in accordance with the invention is a crop plant. By crop plant is meant any plant which is grown on a commercial scale for human or animal consumption or use. In a preferred embodiment, the plant is a cereal, an oilseed plant or a legume.

A plant according to the various aspects of the invention, including the transgenic plants, methods and uses described herein may be a Monocot or a eudicot plant.

### Plants and Crop Species of Interest

The term “plant” as used herein encompasses anything which is capable of undergoing photosynthesis or capable of producing structures which may undergo photosynthesis, along with parts and subcomponents thereof. Common features which undergo or are capable of undergoing photosynthesis include seeds, fruit, shoots, stems, leaves, roots (including tubers), flowers, tissues, and organs. The term “plant” also encompasses plant cells, suspension cultures, callus tissue, embryos, meristematic regions, gametophytes, sporophytes, pollen, and microspores.

A Monocot plant may, for example, be selected from the families Arecaceae, Amaryllidaceae or Poaceae. For example, the plant may be a cereal crop, such as wheat, rice, barley, oat, rye, millet, maize, or a crop such as garlic, onion, leek, yam, pineapple or banana.

A eudicot plant may be selected from the families including, but not limited to Asteraceae, Brassicaceae (e.g. Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from buckwheat, lettuce, sunflower, Arabidopsis, broccoli, spinach, canola, water melon, squash, cabbage, tomato, potato, sweet potato, capsicum, cucumber, courgette, aubergine, carrot, olive, cow pea, hops, raspberry, blackberry, blueberry, almond, walnut, tobacco, cotton, cassava, peanut, sesame, rubber, okra, apple, rose, strawberry, alfalfa, bean, soybean, field (fava) bean, pea, lentil, peanut, chickpea, apricots, pears, peach, grape vine, bell pepper, chilli, flax, camelina, cannabis/hemp, sugar beet, quinoa, citrus, cacao, tea or coffee species. In one embodiment, the plant is oilseed rape (canola).

Also included are biofuel and bioenergy crops such as rape/canola, jute, jatropha, oil palm, linseed, lupin and willow, eucalyptus, poplar, poplar hybrids, or gymnosperms, such as loblolly pine, Norway spruce or sitka spruce. Also included are crops for silage, grazing or fodder (grasses, clover, sanfoin, alfalfa), fibres (e.g. hemp, cotton, flax), building materials (e.g. pine, oak, rubber), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

### EXAMPLES

### Example 1: Transformation of Arabidopsis thaliana with a Genetic Construct for Bundle Sheath Expression of YHB

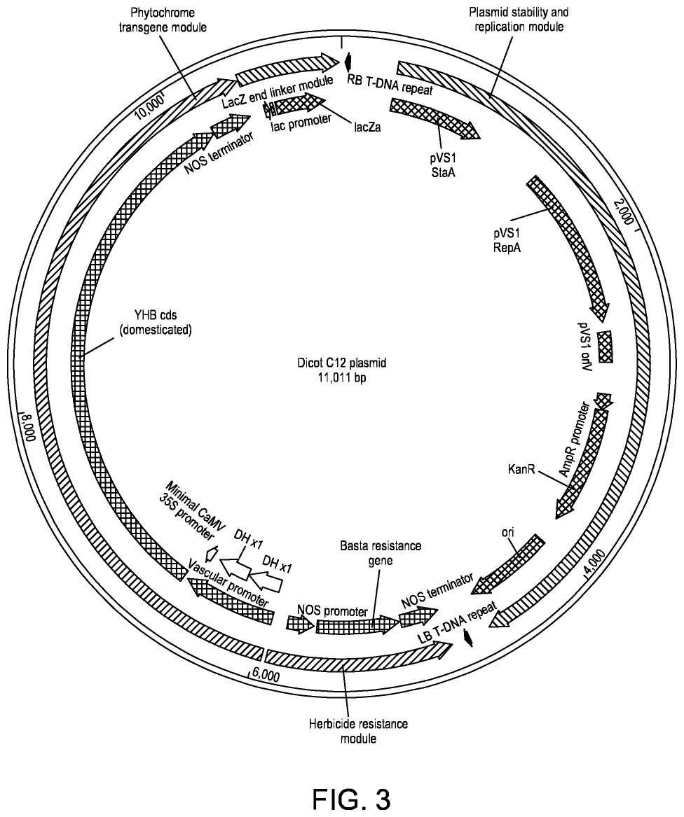

A genetic construct was assembled the Golden Gate cloning system and the resulting plasmid is shown in in FIG. 3. LB and RB refer to Left and Right Borders of the transfer DNA (T-DNA) respectively. The polynucleotide employed by the inventors was the sequence reading LB to RB of a vascular bundle specific promoter, a PHYB variant coding sequence (YHB in this case) and a plant suitable terminator sequence. In total, 6 nucleotide sequence changes were made to the published YHB sequence, none of which changed the corresponding amino acid sequence. These were made to the YHB gene sequence to facilitate the molecular cloning process that assembled the construct. These changes would be unnecessary if this work is replicated by synthesising the construct in a single step, or if alternative cloning strategies were used. Also, whilst construction of this plasmid required the addition of two bacterial marker cassettes, a functionally identical plasmid could be synthesised but without the need for the second bacterial selectable marker cassette that is within the T-DNA region (leftwards of the RB).

As noted above, six nucleotides within the YHB coding sequence [SEQ ID NO: 1] were altered to remove restriction sites prior to gene synthesis, but the amino acid sequence [SEQ ID NO: 4] was unchanged. The DHS vascular bundle specific promoter was used. The DHS promoter sequence [SEQ ID NO: 7] was cloned out of a plasmid first described in Knerova et al., (2018) “A single cis-element that controls cell-type specific expression in Arabidopsis” bioRXiv.). The level two vector contained a herbicide (Basta) resistance cassette and the domesticated YHB genetic sequence downstream of the vascular bundle promoter. Once assembled, the vector was introduced into Agrobacterium tumefasciens (strain AGL-1) cells by electroporation. Agrobacterium colonies that carried the construct were selected on LB plates and cultured on YEB media.

Arabidopsis thaliana (Columbia ecotype) plants propagated in the University of Oxford Department of Plant Sciences were selected at the point of floral emergence (approximately 4 weeks old). Some individuals were set aside and propagated to generate wildtype progeny for use as control plants. The rest were transformed by floral dipping. Once dipped, individuals were grouped into batches of plants to partition seed into independent transformation events. Seeds were sterilised using ethanol and Triton and stratified for three days in a cold room prior to germination. Following germination on soil, T1 plants were screened for transgene insertion by the application of Basta herbicide every other day for one week. T1 transformant plants were transplanted to larger pots and grown to collect T2 seed. T2 seeds were germinated on MS media containing Basta to conduct segregation analysis. Single insertion lines were identified as those that exhibited 75% survival rate on selective media, indicative of a single segregating allele. RNA was extracted from these plants to confirm expression of the YHB transgene in each line. Primers were designed and tested to confirm that they specifically amplified YHB and not native Arabidopsis thaliana Phytochrome B. Three lines representing independent transformation events were selected based on segregation and semi-quantitative PCR results and individual plants from each line were transferred onto soil at 12 days after germination. These were grown alongside wildtype plants in a greenhouse under long day conditions and watered regularly.

All phenotypic analyses in subsequent examples were conducted on all three lines unless otherwise stated, from which comparisons between one transgenic line (annotated as ‘C12’ in the figures) and control plants are displayed in subsequent plots. All error bars indicate 95% confidence intervals and t-tests were used to indicated significance (‘*’) or not (‘n.s.’) at p<0.05.

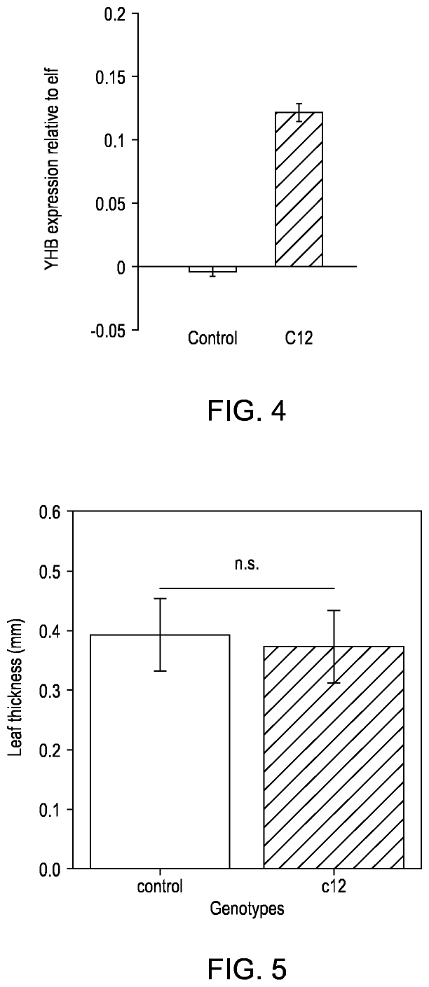

FIG. 4 shows how transfected plants express YHB compared to a house keeping gene called eukaryotic initiation factor eIF-4E1.

Leaf thickness was measured magnetically using a Multispeq V1.0 device. The same leaf (9) was identified for n=10, 5.5 week old plants, measured in three spots near the centre of the leaf and the median value was used for each replicate. As shown in FIG. 5, there was no observable difference in leaf thickness between wildtype controls and transgenic Arabidopsis plants containing the genetic vector for bundle sheath expression of YHB as measured by a t-test (p>0.05). Thus, unlike previous studies that have manipulated the expression of PHYB, the invention described here does not negatively affect leaf thickness.

### Example 2: Expression of YHB in Bundle Sheath Cells Enhances Photosynthetic Capacity in Arabidopsis thaliana

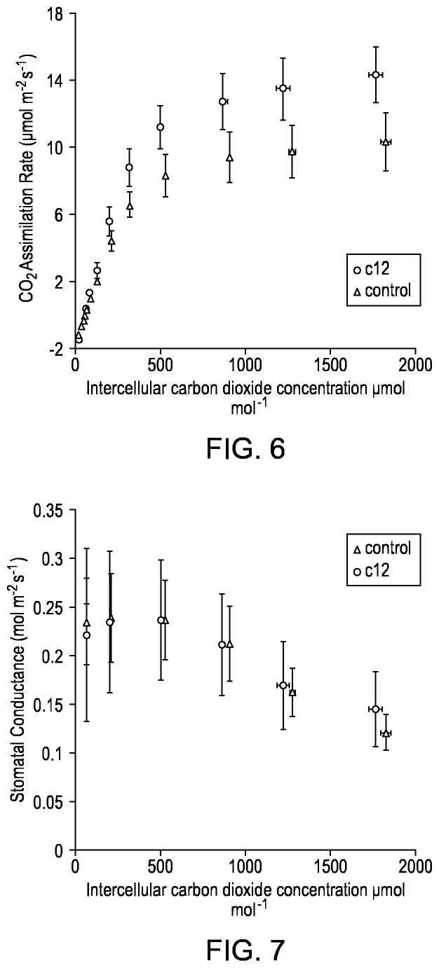

To demonstrate photosynthetic enhancement in transgenic plants compared to non-transformed controls, the plants generated in Example 1 were analysed by gas exchange measurements using a LICOR 6800 device equipped with a multiphase fluorometer head. What is being measured is the amount of carbon that control plants and transgenic Arabidopsis plants containing the genetic vector for bundle sheath expression of YHB could fix given a determined level of ambient carbon dioxide around the leaf (i.e., their photosynthetic rate). Arabidopsis plants growing in a greenhouse were analysed by clamping a leaf in the gas exchange chamber and controlling environmental conditions at 23° C., 65% relative humidity, flow was set to 500 μmol s⁻¹ and fan speed to 10,000 rpm. The same leaf was used for each plant and all plants were measured between 32 to 35 days old, with a mixture of transgenic lines and control plants tested each day between 10 am to 3 pm. Plants were adapted to 400 μmol mol⁻¹ CO₂ and 1500 μmol m⁻¹ s⁻¹ light (with a mixture of 90% red and 10% blue) for 15 minutes, then the carbon dioxide concentration was decreased stepwise from 400 μmol mol⁻¹ to 10 μmol mol⁻¹ then raised back up to 400 μmol mol⁻¹ before increasing to a maximum of 2000 μmol mol⁻¹. Plants were given 5 minutes to acclimate to each new CO₂ concentration then carbon assimilation was measured. Plant leaf area was measured to adjust for slight differences in leaf sizes. The resulting A/Ci curves (FIG. 6) demonstrate significant photosynthetic enhancement in transgenic plants (n=8) when compared to wild type controls (n=12), which manifested as significant increases in maximum photosynthetic capacity, and a significant increase in carboxylation efficiency at lower carbon dioxide concentrations.

Transgenic Arabidopsis plants containing the genetic vector for bundle sheath expression of YHB consistently outperformed controls until carbon dioxide was too low to facilitate photosynthesis in either genotype. The initial slope of these curves show that these transgenic plants have a greater carboxylation efficiency, and the plateauing phase (towards the highest values of carbon dioxide concentrations tested) demonstrate that the maximum photosynthetic rate of these transgenic plants was also increased. Just considering ambient carbon dioxide levels (as would be encountered by crops in fields), what the experiment shows is that these transgenic plants fix significantly more carbon out of the surrounding air than control plants. Thus, unlike previous studies that have manipulated the expression of PHYB, the invention described here substantially improves leaf level photosynthetic rate.

### Example 3: Expression of YHB in Bundle Sheath Cells Enhances Water Use Efficiency in Arabidopsis thaliana

To demonstrate that transgenic Arabidopsis plants containing the genetic vector for bundle sheath expression of YHB showed no negative effects on water use efficiency when compared to control plants, stomatal conductance was measured. This is important, because previous attempts by others to modulate PHYB/YHB expression (e.g., Rao et al., (2011)), have resulted in large increases in water consumption. Stomatal conductance was measured at 400 μmol mol⁻¹ CO₂, 65% relative humidity, 23° C. temperature with flow set to 500 μmol s⁻¹ and a fan speed of 10000 rpm. Importantly, there was no increase in stomatal conductance in transgenic Arabidopsis plants containing the genetic vector for bundle sheath expression of YHB when compared to controls (FIG. 7).

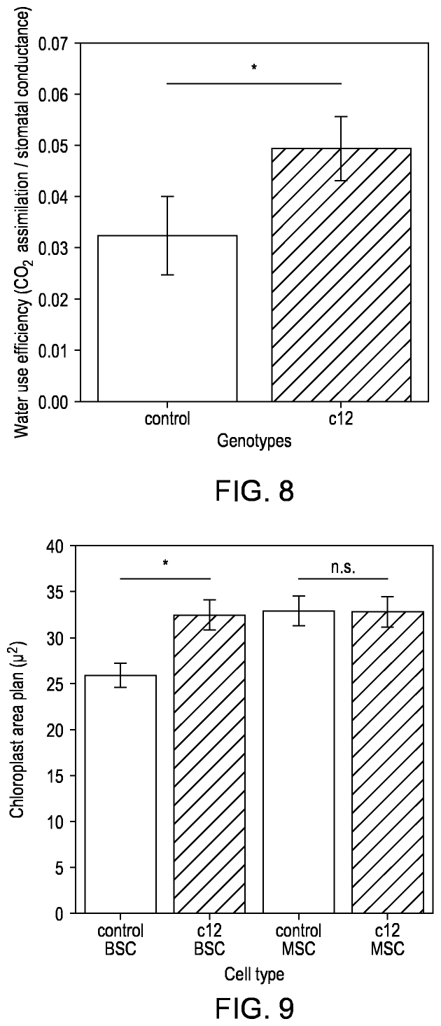

By dividing carbon assimilation rate by stomatal conductance, instantaneous water use efficiency was calculated (carbon captured per water flux). This demonstrated that while photosynthetic rate was at a maximum (as shown in FIG. 6), instantaneous water use efficiency was also significantly increased in transgenic Arabidopsis plants containing the genetic vector for bundle sheath expression of YHB when compared to control plants (see FIG. 8). Thus, water use efficiency was not compromised by the novel photosynthetic enhancement of the invention. Moreover, when photosynthesis is operating at its maximum rate, transgenic Arabidopsis plants containing the genetic vector for bundle sheath expression of YHB had enhanced water use efficiency compared to control plants. Thus, unlike previous studies that have manipulated the expression of PHYB, the invention described here substantially improves leaf level photosynthetic rate while also improving water use efficiency.

### Example 4: Expression of YHB in Bundle Sheath Cells Enhances Chloroplast Development in Bundle Sheath Cells but not Mesophyll Cells in Arabidopsis thaliana

In Arabidopsis leaves, mesophyll cells contain fully developed, photosynthetically active chloroplasts whilst bundle sheath cells contain smaller chloroplasts with reduced photosynthetic capacity. To demonstrate that chloroplasts in bundle sheath cells of transgenic Arabidopsis plants containing the genetic vector for bundle sheath expression of YHB were enhanced compared to control plants, the plants were subject to confocal microscopy and electron microscopy analysis. Equivalent leaves (leaf 6) were harvested from transgenic and control Arabidopsis plants 25 days after germination (as generated in Example 1). The lower epidermis was peeled away, and leaves were fixed in formaldehyde. Once fixed, paradermal sections were placed on a slide and imaged using a confocal microscope. To allocate chloroplasts to particular cell types, both chlorophyll and lignin autofluorescence were imaged in cells surrounding veins. Lignin and chlorophyll autofluorescence were detected by excitation with 458 nm and 633 nm lasers and emission spectra recorded between 465-599 nm and 650-750 nm, respectively. Z stacks were taken around veins to capture mesophyll and bundle sheath cells from a total of five leaves per genotype. For each leaf, five mesophyll and five bundle sheath cells were identified from at least two different images and the chloroplast area plans of the five largest chloroplasts in each cell (i.e. positioned parallel to the Z plane) were calculated using ImageJ. Hence the average chloroplast size per genotype was calculated by measuring a total of 125 chloroplasts across 25 cells distributed between five different plants. Additionally, transmission electron micrographs were obtained by sampling plants at the same time of day (11 am). Tissues were stained and embedded in resin, then thin sections were cut using an ultramicrotome diamond knife. Images were taken on a Siemens transmission electron microscope.

As shown in FIG. 9, the chloroplasts in the bundle sheath cells of transgenic Arabidopsis plants containing the genetic vector for bundle sheath expression of YHB were significantly larger than in the same cells of control plants. In this cell type, YHB expression induced chloroplast development such that these chloroplasts were the same size as mesophyll cell chloroplasts. Mesophyll chloroplasts were unaltered in size between transgenic Arabidopsis plants containing the genetic vector for bundle sheath expression of YHB and controls.

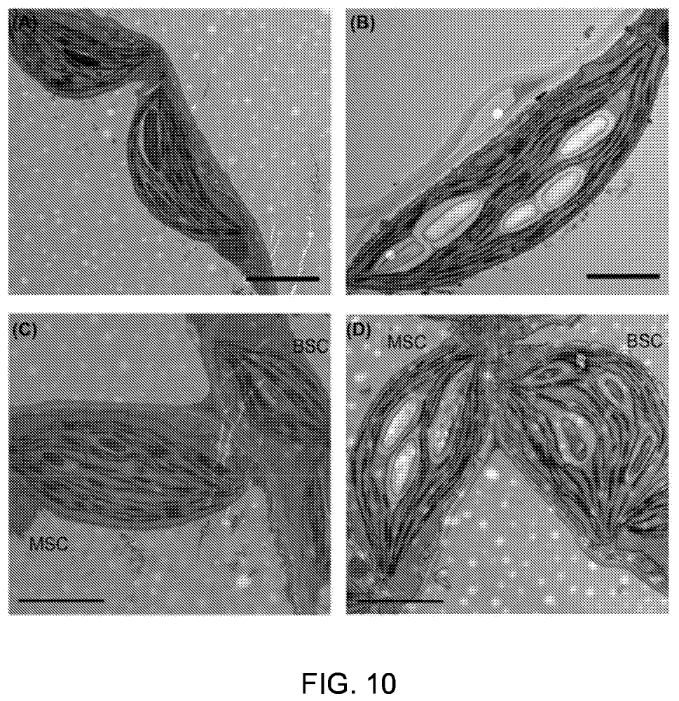

Electron microscopy analysis of transgenic Arabidopsis plants containing the genetic vector for bundle sheath expression of YHB revealed that bundle sheath chloroplasts were equivalent to mesophyll cell chloroplasts in terms of size and organisation of photosynthetic apparatus (FIGS. 10 B and 10 D), whereas bundle sheath chloroplasts in control plants were visibly smaller and less photosynthetically competent compared to mesophyll chloroplasts in the same plants (FIGS. 10A and 10 C). Thus, the invention of the precise expression of YHB in the bundle sheath cells has only effected the chloroplasts of the bundle sheath, and therefore the photosynthetic enhancement described in Example 2 was driven by the photosynthetic activation of bundle sheath cell chloroplasts.

### Example 5: Expression of YHB in Bundle Sheath Cells Enhances Refixation of Respired Carbon Dioxide in Arabidopsis thaliana

Whilst it is the photosynthetic cells that fix CO₂ into sugars, every single plant cell respires, consuming sugars and releasing CO₂. Respiration by cells in the veins releases CO₂, which normally diffuses out of the veins, through the encircling bundle sheath cells, and into the intercellular space where it is either taken back up by the mesophyll or lost from the leaf through stomata. Since transgenic plants showed increased capacity to fix carbon, measurements were made to see if this was in part due to refixation of respired CO₂ by the veins, back into sugars to fuel more growth.

Whilst the CO₂ in the air is comprised of a mixture of carbon-12 and carbon-13 isotopes, the carbon in plant tissues have a signature of less carbon-13 relative to carbon-12 than the air. This is because the enzyme that fixes carbon out of the air, rubisco for C₃ species, discriminates against the heavier carbon-13 isotope, resulting in a negative δ13C ratio as measured by dry matter carbon isotope analysis. If the transgenic Arabidopsis plants containing the genetic vector for bundle sheath expression of YHB (Example 4) were refixing respired carbon (i.e. carbon that had already been fixed once before), then the carbon that ended up in the leaves would be subject to multiple rounds of rubisco mediated fixation and thus multiple rounds of discrimination. Thus, if enhanced refixation of respired CO₂ was occurring in the transgenic plants containing the genetic vector for bundle sheath expression of YHB then one would expect to see a signature of this in carbon isotope analysis. Specifically, one would expect to see a more negative 613C than in equivalent tissues from control plants.

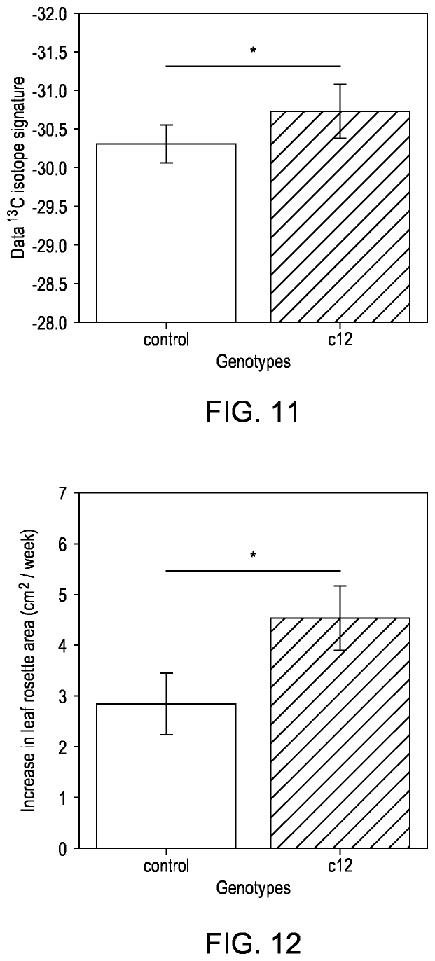

At 36 days old (plants as generated in Example 1) equivalent leaves (leaf 9) were flash frozen in liquid Nitrogen and freeze dried in a lyophiliser for 4 days. Approximately 1 mg of dry leaf powder was weighed out per 6 samples per genotype (two genotypes were tested, one transgenic line and one control group) and subject to stable isotope analysis. This demonstrated that transgenic Arabidopsis plants containing the genetic vector for bundle sheath expression of YHB had a significantly more negative 613C, indicating that respired CO₂ was a significant carbon source in these plants (see FIG. 11). Thus, a component of the photosynthetic enhancement in these plants is attributable to enhanced refixation of respired CO₂. The extent of this refixation enhancement may vary between species, depending on the availability of vascular derived respired/transpired CO₂.

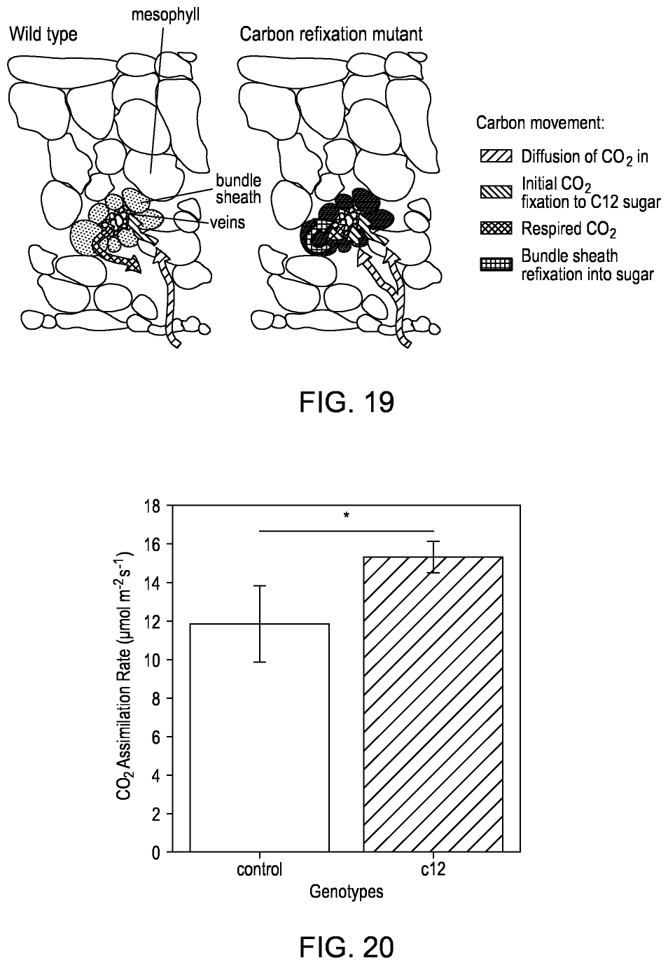

Normal C₃ plants fix the carbon that diffuses into the leaf intercellular space into sugars. Rubisco in photoactivated mesophyll cells fixes the carbon, which is then exported to the vasculature as sugar. These sugars are respired to fuel plant growth throughout the plant. This releases carbon dioxide, which diffuses back out of the veins, around/through bundle sheath cells and out of the leaf. FIG. 19 shows how, in the modified C₃ plants of the invention, initial carbon fixation is carried out primarily by mesophyll cells but the bundle sheath cells of the plants of the invention are also able to do this. Because there are now more active chloroplasts in bundle sheath cells encircling veins, respired carbon dioxide is captured before it can diffuse past the bundle sheath into the intercellular space and out of the plant. This respired CO₂ is therefore re-fixed back into sugars, shifting carbon isotope ratios lower, boosting carbon assimilation efficiency and fuelling more growth per carbon molecule that diffuses into the leaf. Thus, the invention of driving precise expression of YHB only in the bundle sheath cells can also produce the added advantage of enhanced CO₂ refixation.

### Example 6: Expression of YHB in Bundle Sheath Cells Enhances Plant Growth in Arabidopsis thaliana

Given that transgenic Arabidopsis plants containing the genetic vector for bundle sheath expression of YHB had a higher photosynthetic capacity than control plants (Examples 2-5) it was determined how this increase in net carbon uptake might fuel increased plant growth. Photographs were taken from above of trays of 15 plants (as generated in Example 1) at days 14 and 21 after germination. Images were analysed in ImageJ to calculate total rosette area per plant. This showed that, consistent with the increase in photosynthetic rate, the transgenic plants of the invention grew faster than control plants over this time window (FIG. 12).

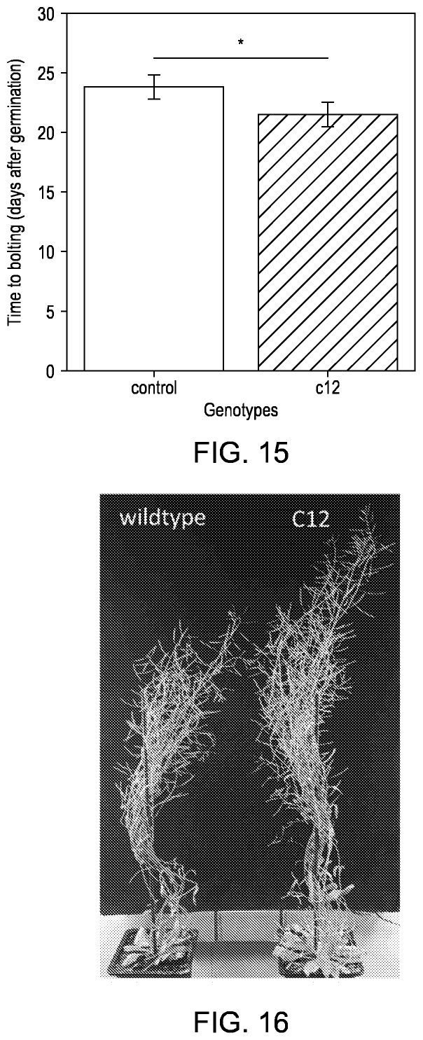

Bolts are the flowering structures of Arabidopsis. Once plants have obtained enough resources during vegetative growth they mature to flowering and invest resources into reproductive structures. Bolting time was measured as the number of days after germination for the plant to grow a bolt that was greater than 3 mm in height. This demonstrated that bolting time is reduced in transgenic Arabidopsis plants containing the genetic vector for bundle sheath expression of YHB compared to wildtype controls (FIG. 15). This is important, as previously described PHYB/YHB overexpressing plants in the literature consistently showed the opposite effect, (i.e. delayed time to bolting/flowering time) across multiple species. Delayed bolting time translates disadvantageously for crop production, as growing seasons are extended and plants lose synchronicity with seasons, and are subject to enhanced risk of loss. This happens because photoactivated PHYB/YHB suppresses Flowering Locus T expression in mesophyll cells, inhibiting flowering. In the present invention this problem is avoided because of no additional expression of PHYB/YHB in mesophyll cells, so flowering time pathways are not interfered with. Thus the reduced time to bolting in the plants of the invention is a novel advantageous trait.

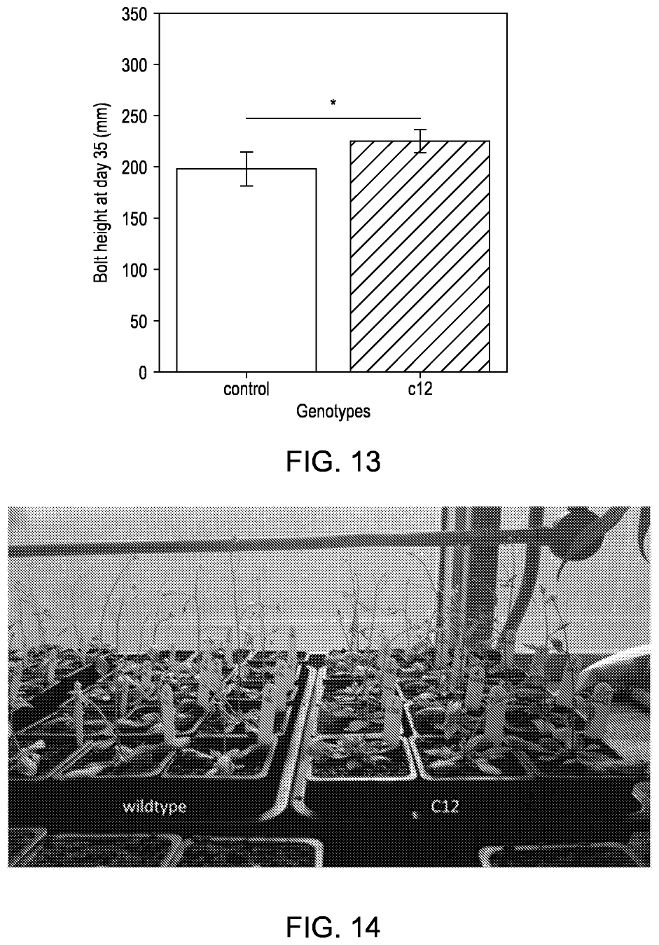

In addition to measuring flowering (bolting) time above, the size of the flowering structure (bolt) was also measured. For each of n=12 plants the tallest bolt was measured at 12 pm using a ruler. Bolts from transgenic Arabidopsis plants containing the genetic vector for bundle sheath expression of YHB were taller than those of control plants 35 days after germination. Rather than being dwarfed, as would be expected given previous work by others in PHYB and YHB overexpression, the inventors found these transgenic plants taller at the same time point (see FIG. 13). The plants otherwise underwent ordinary photomorphogenesis (see FIG. 14, picture of trays). Thus, unlike previous studies that have manipulated the expression of PHYB, the invention described here substantially improves plant growth without any adverse developmental affects expected of PHYB/YHB overexpression.

Thus, the invention of driving precise expression of YHB in bundle sheath cells resulted in faster growth, earlier flowering and larger flowering structures. These are all advantageous traits for agriculture as they mean a shorter growing season, reducing risk of crop lost from adverse weather or pests/pathogens and potentially allowing more harvest cycles per year, something which has added additional value.

### Example 7: Expression of YHB in Bundle Sheath Cells Enhances Yield in Arabidopsis thaliana

Given that the plants of the invention, had higher photosynthetic rates, grew faster, flowered earlier and produced larger flowering structures (FIG. 16). It was investigated whether these advantageous traits produced a corresponding increase in yield.

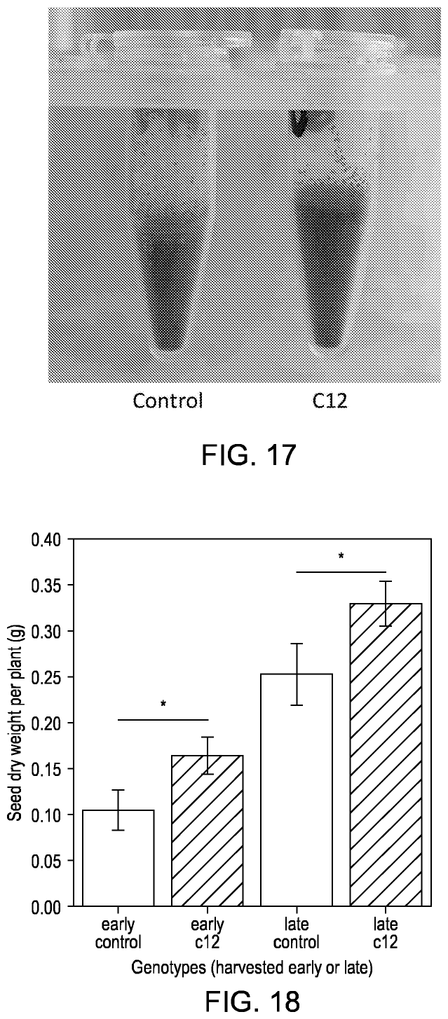

FIG. 17 shows a typical seed harvest for a wildtype plant (left) and a transgenic Arabidopsis plant containing the genetic vector for bundle sheath expression of YHB (right) after watering was stopped at 7.5 weeks, harvesting at 9 weeks and seeds sorted and weighed out at 9.5 weeks. This represents a >30% increase in yield that is statistically significant with T-test statistic<0.0005. This demonstrates how the amount of seed produced per plant is significantly greater in transgenic Arabidopsis plants containing the genetic vector for bundle sheath expression of YHB compared to controls.

Previous experiments on PHYB/YHB overexpression in plants often reports yield enhancement, but this is misleading and would not translate to crop harvests due pleiotropic delays in flowering. For example, Thiele et al., (1999) overexpress PHYB in potato and produce fewer, but more numerous tubers resulting in a reported yield increase. However, they also clarify that this does not occur in the same time frame as conventional potato harvests; and when harvested at the same time as normal potato harvesting, the yield of conventional PHYB overexpressors is lower than controls. Indeed, in Hu et al., (2019) supra, YHB (either derived from Arabidopsis or rice) was overexpressed in a range of diverse species (Arabidopsis, rice, tobacco, tomato and Brachypodium) and YHB overexpression consistently had a negative impact on seed yield. In distinct contrast, the transgenic plants of the inventors show surprisingly a much greater seed yield than controls when harvested at the same time, regardless of the stage at which they are harvested.

Ultimately, transgenic Arabidopsis plants containing the genetic vector for bundle sheath expression of YHB filled these siliques to produce significantly more seed than controls (see FIG. 18), which was consistently enhanced whether seeds were harvested early or late. Here, watering was stopped either ‘early’ at 6.5 weeks old or ‘late’ at 8 weeks old. Plants were allowed to dry for 1.5 weeks before seed harvest. Dry aerial biomass was collected in paper bags and shaken to release seeds. Seeds were sorted out from plant debris using a fine mesh and poured into plastic tubes for weighing. Hence, photosynthetic enhancement was successfully converted into increased yield.

Thus, the invention results in higher photosynthetic rate, enhanced water use efficiency, enhanced CO₂ refixation, faster growth, early flowering, larger flowering structures, and more yield when compared to control plants.

### Example 8: Transformation of Triticum aestivum with a Genetic Construct for Bundle Sheath Expression of YHB

To demonstrate the broad general applicability of this invention and validate the crop enhancement potential of bundle sheath expressed YHB, a monocot optimized plasmid was designed and tested in the monocot crop plant wheat, Triticum aestivum variety Cadenza. Unlike Example 1 which used a synthetic bundle sheath promoter, here the Zoysia japonica phosphoenolpyruvate carboxykinase promoter was used (previously described to provide bundle sheath specific gene expression in monocots (Nomura et al., (2005), Plant Cell Physiol. And FIG. 23). This promoter sequence [SEQ ID 10] is derived from the monocot Zoysia japonica, rather than the eudicot Arabidopsis thaliana. This promoter sequence was designed to drive the expression of an endogenous wheat phytochrome B coding sequence [SEQ ID NO: 11] (Traes_4 AS_1F3163292), which was modified to render it light insensitive through conversion of the amino acid tyrosine at position 278 into histidine—otherwise known as the YHB mutation. The coding sequence of this wheat gene shares 66.11% identity to the Arabidopsis ortholog used in Example 1, and the amino acid sequence [SEQ ID NO: 12] shares 71.28% similarity to the Arabidopsis ortholog, when compared using Clustal 2.1.

The full-length promoter-gene-Nos terminator sequence was fully synthesized de novo. This sequence was integrated into a binary vector containing an nptII selection cassette, transferred into agrobacterium, and used to transform cultured wheat calli using standard plant tissue culture and transformation methods. Transformants were screened to confirm successful genomic insertion and to identify single insertion transgenic plants by qPCR.

Transformants were potted and grown in growth chambers alongside control plants which had been through callus regeneration, but not received the construct for bundle sheath expression of YHB.

### Example 9: Expression of YHB in Bundle Sheath Cells Enhances Photosynthetic Rates in Triticum aestivum

After seven weeks of growth in a growth chamber, photosynthetic rates of transformant wheat plants generated in Example 8 were quantified and compared to control plants. As in Example 2, LICOR 6800 devices were used to accurately measure photosynthetic rate. Environmental constants were as follows: flow 500 μmol s⁻¹, fan speed 10,000 rpm, leaf temperature 25° C., 65% relative humidity. To measure the ambient photosynthetic rate in the growth chambers, PAR (photosynthetically active radiation, i.e. the amount of light available for photosynthesis) was set to 350 μmol m⁻¹ s⁻¹ (which was the measured light intensity at canopy height in the growth chamber) and carbon dioxide to 400 μmol mol⁻¹. For each plant, the leaf below the flag leaf was selected and clamped ˜⅓ from the leaf tip. Following 10 minutes of acclimation (confirmed by observing no change in assimilation rate, fluorescence or stomatal conductance following this acclimation), an ambient photosynthetic measurement was recorded. Four controls and eight single insert wheat plants were screened between 12:00-14:00 on the same day. FIG. 20 shows the result of this analysis: Photosynthetic rate was on average 30% higher in wheat plants containing the genetic vector for bundle sheath expression of YHB compared to controls t-test at p<0.05.

### Example 10: Expression of YHB in Bundle Sheath Cells Enhances Growth Rates in Triticum aestivum

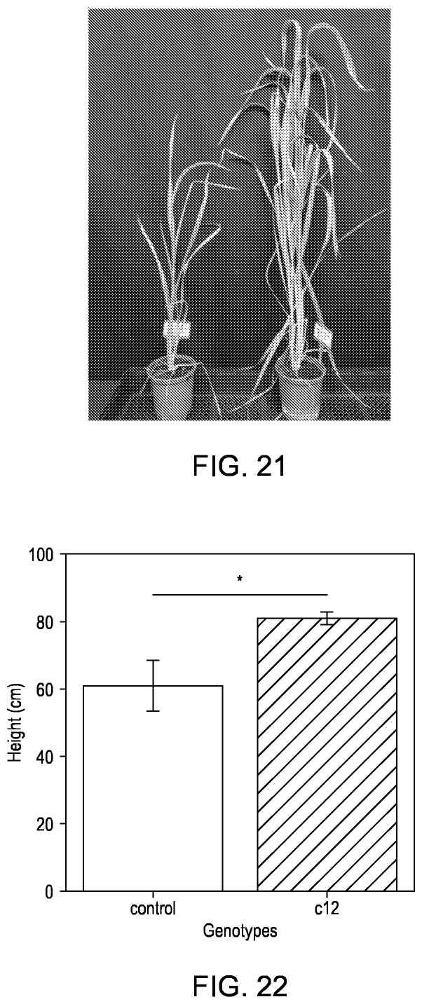

As demonstrated in Example 6, enhanced Phytochrome B signalling in the vascular bundles of Arabidopsis was associated with faster growth, indicated by increase biomass accumulation compared to controls in the same time window, but not with changes to overall development of plant architecture. Likewise, wheat plants containing the genetic vector for bundle sheath expression of YHB showed no changes in development (such as dwarfing) and normal flowering was observed. As indicated by FIG. 21, typical wheat plants containing the genetic vector for bundle sheath expression of YHB were significantly larger than controls after seven weeks of growth.

Indeed, plant height (as measured as maximum canopy height, from soil surface to tip of tallest point) was significantly higher (by t-test, at p<0.05) in transformants (n=8) than controls (n=4) (FIG. 22). At this time point, the transformants had appeared to reach full height and started flowering while the controls were still ˜⅔ this maximum height. This ˜30% faster growth was primarily attributed to the 30% increase photosynthetic rate observed in Example 9. Hence, despite the extensive genetic differences and evolutionary distance between the eudicot Arabidopsis thaliana and monocot Triticum aestivum, this invention consistently enhances photosynthesis, does not disrupt development, and enhances plant productivity.

A person of average skill in the art could therefore combine any promoter sequence known to activate vascular bundle expression (either those known in literature or by designing a new promoter), and over express either an endogenous Phytochrome B gene or an exogenous Phytochrome B gene or YHB variant or functional fragment thereof to apply this invention to any desired crop. Likewise, various transformation methods can be used (whether floral dipping as in Example 1 or callus transformation as in this example) depending on species of interest.

### Example 11: Gene Editing Brassica napus for Bundle Sheath Expression of PHYB and or YHB

As noted in FIG. 2 and FIG. 25, PHYB has duplicated in a number of agronomically important species, such as Brassica napus and Glycine max. In fact, most of our crops have experienced recent whole genome duplication events and contain multiple redundant copies of PHYB. This means that it is possible to convert one copy of PHYB into a vascular-bundle driven YHB while the other copy is unaffected. This would have the same result on the plant as introducing YHB through genetic modification (as Examples 1 and 8), but would not require the addition of any transgenic material and therefore result in a gene edited plant instead. This has the additional benefit of ensuring that native PHYB signalling is not removed, which would otherwise result in developmental defects in planta.

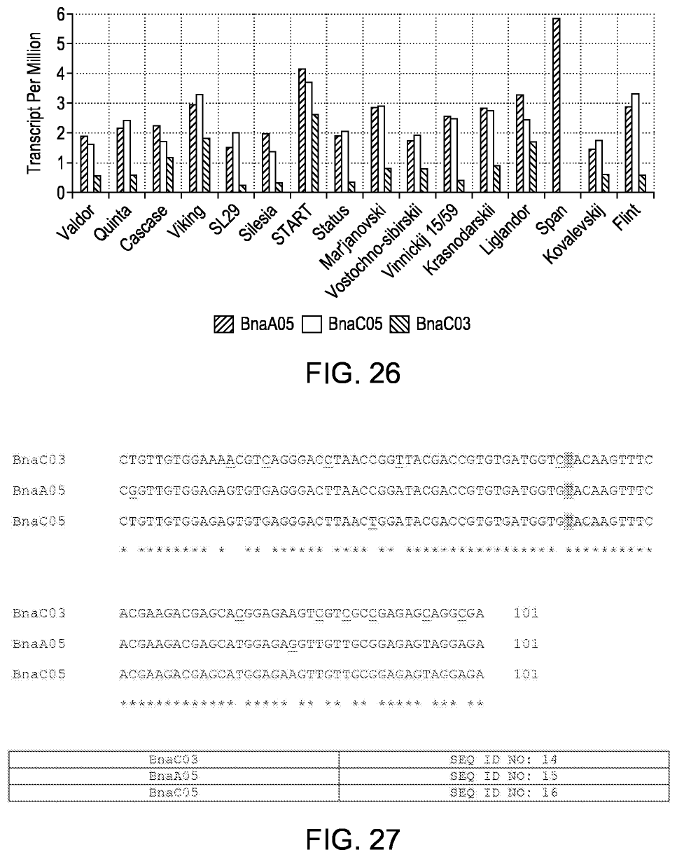

Brassica napus provides an example species where genome editing may be used to achieve bundle sheath expression of YHB using standard genome editing technologies known to a person of average skill in the art. FIG. 26 shows the expression of the three PHYB genes encoded in the B. napus genome (BnaA05g22950D, BnaC05g36390D and BnaC03g39830D, hereafter referred to as BnaA05, BnaC05 and BnaC03, respectively) in the leaves of 16 distinct cultivars of this crop species (RNA was sampled from the second youngest leaves when plants were at the five true leaf stage, Hong et al., (2019) Nat. Comms. 10:2878). PHYB homologs BnaA05 and BnaC05 are expressed in the leaves of all varieties, and both are expressed to the same extent in each variety, providing evidence that they function redundantly. The exception to this pattern is the Span cultivar, in which BnaC05 is not expressed. However, given that Span undergoes normal photosynthetic development, this is further evidence that both PHYBs act redundantly i.e. expression of BnaA05 compensates for a lack of expression of BnaC05. Thus, it will be possible to engineer one variant for the purposes of photosynthetic enhancement without disrupting normal photomorphogenesis.

Initially, the gene expression domain of a native PHYB gene would be changed such that it was expressed in the vascular bundle. This would be achieved through a knock in of a short promoter sequence (e.g. SEQ ID: 7) or any vascular bundle or bundle sheath promoter or bundle sheath enhancer element known to a person of average skill in the art into the 5′ upstream region of a native PHYB gene (e.g. BnaA05). The bundle sheath promoters hereinbefore described and also illustrated in FIG. 23 work over large phylogenetic distances (90-160 million year divergence times). GLDP and SULTR2;2 give consistent expression patterns in Arabidopsis and Flaveria, representing deep conservation between Rosids and Asterids, which diverged ˜125 million years ago. Flaveria is equally related to B. napus as it is to A. thaliana, and so promoters that work in both Flaveria and Arabidopsis are expected to work in B. napus as well. The MYB76 regulatory element used in Example 1 has been shown to be highly conserved between Arabidopsis and Brassica genera, are they are closely related (having diverged just ˜20 million years ago). Many promoters are available for the person of average skill to choose from for directing expression of native PHYB genes.

In accordance with the invention, the editing of the native PHYB gene which inserts a vascular sheath promoter results in expected expression of PHYB in the requisite tissue. A stably inherited PHYB sequence is functionally equivalent to the polynucleotide integrated into the Arabidopsis or wheat genomes as described in Example 1 and Example 8. Any region in the 5′ upstream region may be a suitable target site for knocking in these promoter sequences. An endonuclease would be directed to a specific site to induce a double strand DNA break, and homology arms would direct the promoter polynucleotide to this area, to be incorporated into the DNA by homology directed repair. This has already been demonstrated in plants with suitable efficiency. For example, CRISPR-Cpf1 has been used to knock in >3,000 bp pieces of DNA into the rice genome with 8% efficiency (Begemann et al., (2017) Sci. Reps. 7:11606). Given that the vascular bundle promoter element is much shorter than this example, and shorter sequences result in higher knock in efficiency, this knock in will be feasible without further inventive steps. B. napus can be transformed using Agrobacterium (as Example 1) and independent transformation events screened by PCR to find individuals in which the promoter element has successfully been incorporated upstream of PHYB. Plants descended from these individuals would have enhanced PHYB expression in the vascular bundles, which can be tested by gene expression analysis, and would be expected to show some enhanced chloroplast development, photosynthetic rates and productivity, without the developmental defects associated with altering PHYB expression at the whole plant level (such as the semi-dwarfing phenotype that results from ubiquitous overexpression of PHYB). The phenotype would be expected to be analogous to that described in this document from introduction of vascular bundle expressed PHYB using conventional genetic modification approaches.

To further amplify PHYB signalling activity in the vasculature of B. napus, it may also be necessary to make a second edit, to convert the vascular-bundle driven PHYB into YHB. This too could be delivered with gene editing, but only requires a point mutation rather than a double strand DNA break. In Arabidopsis PHYB, a TAT′ codon is changed into ‘CAT’ to convert residue 276 from tyrosine to histidine and change PHYB into YHB. For BnaA05, a ‘TAO’ codon encodes the equivalent tyrosine residue, which can be changed into ‘CAC’ to make the equivalent modification to histidine by the introduction of a single nucleotide change. FIG. 27 illustrates the region of the B. napus PHYB coding sequences in which this single base pair change can be made [SEQ ID NOs: 14, 15 & 16]. This edit can be brought about by a nickase e.g. Cas9, tethered to an adenosine deaminase; the nuclease creates a small window of single stranded DNA which directs the deaminase to a specific section of DNA to convert adenine to guanine. This type of editing has previously been demonstrated in Arabidopsis plants and B. napus protoplasts, with up to 8.8% efficiency in the latter species (Beum-Chang Kang et al., (2018) Nat. Plants. 4:427-431). By targeting the reverse strand of a PHYB gene, this system would be sufficient to induce the adenine to guanine conversion that results in a complementary conversion of thymine to cytosine on the forward strand, thereby shifting the codon from ‘TAC’ to ‘CAC’ and therefore, PHYB to YHB. This T to C mutation could also be readily achieved by prime editing (Anzalone et al. (2020) Nature Biotechnology, 38:824-844), or by random targeted mutagenesis at the correct site by CRISPR-Cas or other genome editing nucleases through techniques known to a person of average skill in the art.

As indicated by FIG. 27, despite high conservation in the nucleotide sequences of the multiple copies of the PHYB gene in the B. napus genome, each homolog contains multiple unique variations which can be used to direct targeted base editing to a specific gene variant i.e. only the PHYB gene whose expression domain was previously edited, thereby ensuring that YHB expression is restricted to the vascular bundles. Transformed plants would be screened by PCR to find individuals containing this YHB edit, and it would be expected that any increase to photosynthesis and productivity that was previously induced by the first change may be further amplified by this second change.

Both of the genome edits proposed here have been demonstrated in planta to high levels of efficiency, even in species that are hard to transform and require methods other than floral dip, such as callus regeneration or particle bombardment. Thus, this B. napus example provides a general methodology for introducing vascular bundle expressed YHB through genome editing, in any species containing more than one copy of PHYB. Moreover, this approach can also be taken in any diploid plant so long as the transformants were maintained as heterozygous plants containing one unaltered copy of the PHYB allele and one altered copy of the PHYB allele. In summary, a single copy of PHYB is targeted for editing using nucleotide variation that is specific to that copy. In the first instance, PHYB expression is enhanced in the vascular bundles by knocking in a vascular sheath or vascular bundle specific promoter into the 5′ upstream region. This same gene is then subsequently targeted for a single nucleotide mutation in the CDS (coding sequence); the codon encoding a tyrosine residue that gives native PHYB the ability to revert from its photoactive form is mutated into histidine. This converts the native PHYB into constitutively active YHB, which further enhances PHYB signalling cascades in the vascular bundle. It is worth noting that even in species that lack a redundant copy of PHYB, it would even be possible to knock in a full length PHYB copy first, thereby creating a copy that can be gene edited further. Notably, all of these gene editing proposals achieve the same end result that was demonstrated in Example 1 and Example 8 by genetic modification methodology: A PHYB homolog that is expressed in vascular sheath cells.

Finally, the effects of altering PHYB expression (by knock out or overexpression) are highly conserved between distantly related species (FIG. 24), and multiple promoters derived from different, distantly related species enable vascular sheath expression to be driven in across the breadth of vascular plants (FIG. 23). The illustrative examples provided herein are understood to be exemplary, such that a person of average skill in the art can deliver this trait in any vascular plant species through any one of the genetic engineering methods described above.

### Example 12: Generalised Gene Editing Protocol for Activating Bundle Sheath Expression of PHYB and or YHB in any Plant Species

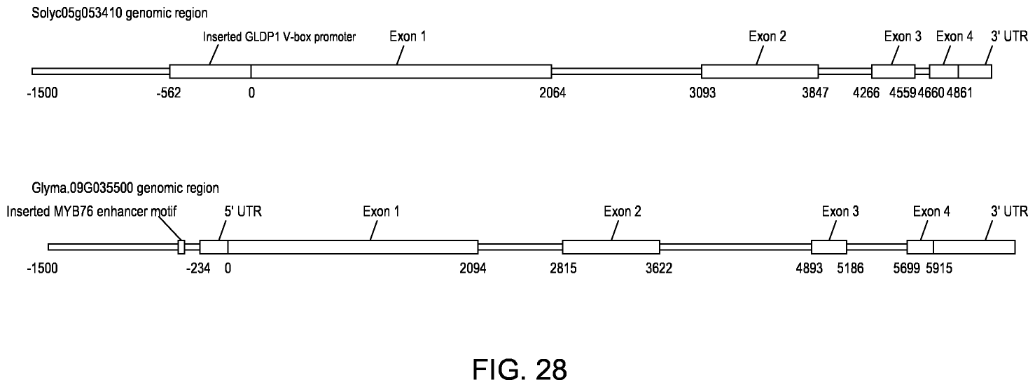

In addition to the full promoter knock-in example of Example 11, it is also possible to restrict the size of the gene edit to a few base pairs by only introducing a small vascular sheath or vascular bundle motif or enhancer element into the promoter region of endogenous PHYB genes. FIG. 28 provides a comparison between these two approaches, demonstrated with designs for tomato (Solanum lycopersicum) and soybean (Glycine max) in which proposed gene edits have been annotated on genome models for a PHYB ortholog in the former (Solyc05g053410) and latter (Glyma.09G035500) species. The Glycine Decarboxylase P subunit (GLDP) promoter has been characterised in Asterid Flaveria bidentis and in Rosid Arabidopsis thaliana. A deletion series revealed that the V-box containing GLDP1 promoter region is sufficient to drive vascular bundle expression (Adwy et al., (2015) The Plant Journal. 84(6):1231-1238). Thus, in tomato, vascular bundle expression of PHYB could be introduced by knocking in the GLDP1 V-box containing promoter [SEQ ID NO: 13] immediately upstream of the first exon of the endogenous PHYB gene identified here, using similar methods to those discussed in Example 11 (as shown in FIG. 28, top image). i.e. a person of average skill in the art could use such designs to target a variety of genome editing nucleases to the target locus with a DNA repair template encoding the promoter sequences of choice, and generate gene edited plants.

The MYB76 promoter used in Example 1 has also been shown to drive tissue specific gene expression through the action of a small minimal enhancer motif (Dickinson et al., (2020) Nature Plants. 6:1468-1479). Such an enhancer motif sequence could be introduced in close proximity to the transcription start site of soybean's PHYB gene to confer the desired expression pattern. Unlike the tomato design described above, this approach would leave the endogenous core promoter intact, as indicated in FIG. 28 by the presence of native 5′ UTR (bottom image). Core promoters can be further characterised through a variety of commonplace techniques, including but not limited to TSS-seq, CAP-seq, and CHIP-seq to identify open chromatic regions. This additional characterisation would help to identify the exact locus where RNA polymerase binds to initiate transcription, thereby ensuring that the exact genomic location within which the vascular bundle enhancer motif is inserted will not disrupt this region (though it would also be possible to simply try several locations out and confirm success with gene expression analyses in transgenic plants). Hence, this enhancer element insertion method would enable editing of native PHYB genes without disrupting native expression pattern, and enable editing of PHYB expression profiles in species that only have one copy of this gene. Given these advantages, it may be preferable to further shrink known vascular bundle promoters e.g. the GLDP1 V-box, into minimal enhancer sequences that can be introduced by editing as few bases as possible, e.g. by using the same molecular methods that have already been published in the case of reducing the full length MYB76 promoter into a necessary and sufficient minimal enhancer motif sequence (Dickinson et al., (2020) Nature Plants. 6:1468-1479). Subsequent conversion of the bundle sheath expressed PHYB to YHB as described in Example 11 can optionally be conducted to further enhance PHYB signalling the bundle sheath cells. This single nucleotide mutation could also be readily achieved by base editing, prime editing, or by random targeted mutagenesis at the correct site by CRISPR-Cas or other genome editing nucleases through techniques known to a person of average skill in the art.

### Genetic Resources

Seeds of Arabidopsis thaliana (Columbia ecotype) were obtained in September 2018 from the University of Oxford Department of Plant Sciences greenhouses.

Golden gate cloning parts were provided by Sylvestre Marillonnet (Liebnitz Institute of Plant Biochemistry: Weber et al., (2011) PLOS ONE). The DHS vascular bundle promoter was provided by Patrick Dickinson from Julian Hibberd's lab, Cambridge University (Kneřová et al., (2018) bioRxiv).

Cadenza wheat plants and wheat transformation was provided by NIAB Crop Transformation Services.

### Nucleotide and Amino Acid Sequences

[SEQ ID NO: 1] The domesticated Arabidopsis thaliana PHYB coding sequence, containing the YHB mutation.

[SEQ ID NO: 2] The domesticated Arabidopsis thaliana PHYB coding sequence (Arabidopsis_PHYB_AT2G18790.1).

[SEQ ID NO: 3] The rice PHYB coding sequence (Rice_PHYB_LOC_Os03g19590.1).

[SEQ ID NO: 4] The Arabidopsis thaliana YHB amino acid sequence.

[SEQ ID NO: 5] The Arabidopsis thaliana PHYB amino acid sequence (Arabidopsis_PHYB_AT2G18790.1).

[SEQ ID NO: 6] The rice PHYB amino acid sequence (Rice_PHYB_LOC_Os03g19590.1).

[SEQ ID NO: 7] The nucleotide sequence of the Arabidopsis derived MYB76 vascular bundle promoter. This is a synthetic promoter comprised of an oligomerised MYB76 sequence containing a minimal enhancer element, and a 35S minimal core promoter element.

[SEQ ID NO: 8] Arabidopsis thaliana phytochrome D nucleotide coding DNA sequence (Arabidopsis_PHYD_AT4G16250.1).

[SEQ ID NO: 9] Arabidopsis thaliana phytochrome D amino acid sequence (Arabidopsis_PHYD_AT4G16250.1).

[SEQ ID NO:10] The Zoysia japonica PCK promoter sequence.

[SEQ ID NO:11] The wheat PHYB coding sequence containing the YHB mutation (derived from Traes_4 AS_1F3163292).

[SEQ ID NO:12] The wheat PHYB amino acid sequence containing the YHB mutation (derived from Traes_4 AS_1F3163292).

[SEQ ID NO:13] The GLDP1 V-box containing promoter DNA sequence.

[SEQ ID NO: 14] Brassica napus PHYB coding sequence excerpt (BnaC03g39830D).

[SEQ ID NO: 15] Brassica napus PHYB coding sequence excerpt (BnaA05g22950D).

[SEQ ID NO: 16] Brassica napus PHYB coding sequence excerpt (BnaC05g36390D).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of increasing photosynthetic capacity of a C₃ plant, the method comprising altering heritable genetic material of the C₃ plant such that a gene of interest (GOI) is expressed in at least one vascular sheath cell of the C₃ plant, and wherein the GOI is expressed under the control of a gene expression regulatory element active in the at least one vascular sheath cell of the C₃ plant.

2. The method as claimed in claim 1, wherein the GOI encodes phytochrome B, an active variant thereof, or functional fragment thereof.

3. The method as claimed in claim 1 or claim 2, wherein the gene expression regulatory element is active specifically in the at least one vascular sheath cell of the C₃ plant.

4. The method as claimed in any of claims 1 to 3, wherein the altering of the heritable genetic material comprises inserting at least one polynucleotide into the heritable genetic material of a cell of the C₃ plant.

5. The method as claimed in any of claims 1 to 4, wherein the altering of the heritable genetic material comprises the use of a base editor; optionally a prime editor.

6. The method as claimed in any of claims 1 to 4, wherein the altering of the heritable genetic material comprises introducing a gene repair oligonucleobase (GRON)-mediated mutation into a target DNA sequence of the heritable genetic material of a cell of the C₃ plant; optionally exposing the cell of the C₃ plant to a DNA cutter and a GRON.

7. The method as claimed in claim 6, wherein the DNA cutter comprises a meganuclease, a transcription activator-like effector nuclease (TALEN), a zinc finger, an antibiotic, or a Cas protein.

8. The method as claimed in any of claims 1 to 3, wherein the altering of the heritable genetic material comprises using zinc finger nucleases (ZNFs) and/or transcription activator-like effector nucleases (TALENs) for site-specific homologous recombination of the heritable genetic material of a cell of the C₃ plant.

9. The method as claimed in any of claims 1 to 3, wherein altering of the heritable genetic material comprises introducing a donor template to the heritable genetic material of a cell of the C₃ plant using a viral vector.

10. The method as claimed in claim 9, wherein the viral vector comprises a protein expression vector; optionally wherein the protein expression vector comprises pQE or pET.

11. The method as claimed in any of claims 1 to 4, wherein the one or more polynucleotides comprises a polynucleotide encoding a CRISPR-Cas protein, optionally a guide RNA (gRNA), and a donor polynucleotide comprising a sequence of the gene expression regulatory element, wherein the gRNA directs the CRISPR-Cas protein to the locus of at least one copy of the GOI in the genome of a cell of the C₃ plant, whereby the gene expression regulatory element is inserted so as to cause expression of the copy or copies of the GOI in the at least one vascular sheath cell of a plant regenerated from the cell.

12. The method as claimed in claim 11, wherein the CRISPR-Cas protein and the gRNA are preassembled to form ribonucleoproteins (RNPs); optionally wherein the RNPs are transfected into the cell.

13. The method as claimed in claim 11 or claim 12, wherein the RNPs are transfected into the cell using electroporation.

14. The method as claimed in any of claims 11 to 13, wherein the CRISPR-Cas protein comprises Cas9, Cas12a, or Cas 12b.

15. The method of any of claims 11 to 13, wherein the polynucleotide encoding a CRISPR-Cas protein is introduced via a plasmid.

16. The method as claimed in claim 4, wherein at least one polynucleotide comprises the expression regulatory element, a nucleotide sequence which encodes the GOI, and optionally a terminator; and a further polynucleotide encodes a CRISPR-Cas protein, and the further polynucleotide or an additional further polynucleotide optionally encodes a gRNA which directs the CRISPR-Cas protein to a desired locus in the genome of the C₃ plant, such that an heterologous GOI under control of the vascular sheath regulatory element is inserted into the desired locus in the cell of the C₃ plant.

17. The method as claimed in claim 16, wherein the at least one polynucleotide comprises from 5′ to 3′ the expression regulatory element, the nucleotide sequence encoding phytochrome B, or active variant thereof, or functional fragment thereof, and optionally the terminator.

18. The method as claimed in claim 4, wherein at least one polynucleotide comprises from 5′ to 3′, the expression regulatory element active specifically in at least some vascular sheath cells of a C₃ plant, a nucleotide sequence which encodes a phytochrome B, or active variant thereof, or functional fragment thereof, such that the phytochrome B or active variant thereof or functional fragment thereof is inserted into the genome of the C₃ plant.

19. An isolated DNA polynucleotide comprising from 5′ to 3′, an expression regulatory element active specifically in at least some vascular sheath cells of a C₃ plant, a nucleotide sequence which encodes a phytochrome B or active variant thereof or a functional fragment thereof, and optionally a terminator.

20. The isolated DNA polynucleotide as claimed in claim 19, wherein the regulatory element comprises a promoter.

21. The isolated DNA polynucleotide as claimed in claim 19 or claim 20, wherein the promoter is a bundle sheath cell-specific promoter and/or a mestome sheath specific promoter or a promoter that is active throughout the vascular bundle.

22. The isolated DNA polynucleotide as claimed in any of claim 21, wherein the bundle sheath specific promoter or the mestome sheath specific promoter or the promoter that is active throughout the vascular bundle is a synthetic promoter; preferably comprised of a bundle sheath or a mestome sheath specific transcription factor binding element upstream of the promoter; optionally wherein there are two or more transcription factor binding elements.

23. The isolated DNA polynucleotide as claimed in any of claims 21 to 22, wherein the bundle sheath specific promoter or the mestome sheath specific promoter or the promoter that is active throughout the vascular bundle is selected from a minimal ZmUbi1 promoter, a NOS core promoter, a CHSA core promoter, and a minimal 35S promoter; preferably wherein the promoter has a nucleotide sequence of SEQ ID NO: 7, or SEQ ID NO: 10, or SEQ ID NO: 13 or a sequence of at least 80% identity therewith.

24. The isolated DNA polynucleotide as claimed in any of claims 21 to 234, wherein the bundle sheath specific promoter or mestome sheath specific promoter or the promoter that is active throughout the vascular bundle is derived from a bundle sheath specific gene or a mestome sheath specific gene, respectively.

25. The isolated DNA polynucleotide as claimed in any of claims 21 to 24, wherein the bundle sheath specific gene is from a plant species; including but not limited to: Arabidopsis thaliana MYB76, Flaveria trinervia GLDP, Arabidopsis thaliana SULTR2;2, Arabidopsis thaliana SCR, Arabidopsis thaliana SCRL23, Zoysia japonica PCK, Urochloa panicoides PCK1 and Hordeum vulgare PHT1;1.

26. The isolated DNA polynucleotide as claimed in any of claims 19 to 25, wherein the promoter is derived from non-plant organisms, such as a rice tungro bacilliform virus (RTBV) promoter.

27. The isolated DNA polynucleotide as claimed in any of claims 19 to 26, wherein the nucleotide sequence which encodes a phytochrome B is any of SEQ ID NO: 1, SEQ ID NO: 2, SEQ ID NO: 3 or SEQ ID NO: 8, or SEQ ID NO: 11, or a sequence of at least 65% identity with any of the sequences, or a functional fragment thereof; preferably a sequence of at least 70% identity with any of the sequences, or a functional fragment thereof; more preferably a sequence of at least 80% identity with any of the sequences, or a functional fragment thereof.

28. The isolated DNA polynucleotide as claimed in any of claims 19 to 27, wherein the functional fragment of the phytochrome B has phytochrome signalling activity, but lacks light sensitivity; preferably wherein the functional fragment consists of the PAS and GAF domains.

29. The isolated DNA polynucleotide as claimed in any of claims 19 to 28, wherein the phytochrome B is light insensitive; preferably YHB and the nucleotide sequence which encodes the phytochrome B is SEQ ID NO: 1, or a sequence of at least 70% identity therewith or a functional fragment thereof.

30. A plasmid comprising a DNA polynucleotide of any of claims 17 to 29, an origin of replication, a T-DNA right border repeat of a Ti or Ri plasmid; optionally additionally a left border repeat of a Ti or Ri plasmid, and at least one bacterial selectable marker.

31. The plasmid as claimed in claim 30, further comprising an element selected from one or more of: an enhancer, a plant selectable marker, a multicloning site, or a recombination site.

32. A Ti or Ri plasmid comprising the DNA polynucleotide of any of claims 17 to 29.

33. A composition for transformation of plant cells comprising the isolated DNA polynucleotide of any of claims 19 to 29, or a plasmid of any of claims 30 to 32; optionally comprising microparticles coated with said DNA polynucleotide or said plasmid.

34. A bacterium comprising the isolated DNA polynucleotide of any of claims 19 to 29, or a plasmid of any of claims 30 to 32; optionally wherein the bacterium is E coli.

35. A bacterium comprising a plasmid of any of claims 30 to 32; preferably wherein the bacterium is Agrobacterium sp.; more preferably A. tumefaciens.

36. A plant which carries out C₃ photosynthesis in at least a part thereof, the plant comprising the isolated DNA polynucleotide of any of claims 19 to 29 stably integrated into the genome thereof; preferably heritably integrated into the genome thereof.

37. A plant which carries out C₃ photosynthesis in at least a part thereof, wherein the plant has an additional at least one additional copy of a phytochrome B gene or functional fragment thereof, and wherein the plant is genetically altered compared to a genetically equivalent unaltered plant, wherein an expression regulatory element(s) of at least one copy of a phytochrome B gene or functional fragment thereof in the altered plant causes an additional at least one phytochrome B gene, or functional fragment thereof, expression specifically in at least some bundle sheath cells and/or mestome sheath cells and/or vascular bundle of the plant compared to the unaltered plant.

38. The plant as claimed in claim 37, wherein the expression regulatory element is a promoter which is active specifically in the at least some vascular sheath cells of a C₃ plant.

39. The plant as claimed in claim 37 or claim 38, wherein the coding sequence of the additional at least one phytochrome B gene is the same as a native phytochrome B gene or genes in the plant.

40. The plant as claimed in claim 37 or claim 38, wherein the additional at least one phytochrome B gene is different to the native phytochrome B gene or genes in the plant; optionally wherein the phytochrome B or active variant or functional fragment thereof is defined in any of claims 27 to 30.

41. The plant as claimed in any of claims 37 to 40 obtained by a process of CRISPR-Cas protein genetic modification.

42. The plant as claimed in any of claims 37 to 41, wherein the genetic modification is heritably stable.

43. The plant as claimed in any of claims 36 to 42 which is a C₃ plant; preferably a crop plant, e.g. a cereal crop plant, an oilseed crop plant or a legume.

44. The plant as claimed in any of claims 37 to 43, wherein the phytochrome B has an amino acid sequence of any of SEQ ID NO: 4, SEQ ID NO: 5, SEQ ID NO: 6, or SEQ ID NO: 9, or SEQ ID NO: 12, or a sequence of at least 65% identity with any of the sequences or a functional fragment thereof; preferably a sequence of at least 70% identity with any of the sequences or a functional fragment thereof; more preferably a sequence of at least 80% identity with any of the sequences or a functional fragment thereof.

45. The plant as claimed in any of claims 37 to 44, wherein the functional fragment of the phytochrome B has phytochrome signalling activity, but lacks light sensitivity;
preferably wherein the fragment consists of the PAS and GAF domains.

46. The plant as claimed in any of claims 37 to 45, wherein the phytochrome B is a light insensitive sequence variant or functional fragment thereof; preferably YHB with an amino acid sequence of SEQ ID NO: 4, SEQ ID NO: 12 or a sequence of at least 70% identity therewith or functional fragment thereof.

47. The plant as claimed in any of claims 37 to 46, wherein the chloroplasts present in vascular sheath cells are developmentally enhanced, in terms of size or photosynthetic capacity, compared to chloroplasts in equivalent vascular sheath cells of control unmodified plants grown under the same conditions for the same period of time.

48. The plant as claimed in any of claims 36 to 47, wherein photosynthesis is enhanced compared to a control unmodified plant grown under the same conditions.

49. The plant as claimed in any of claims 36 to 48, wherein leaf photosynthetic efficiency is greater than in the equivalent leaf or leaves of a control unmodified plant grown under the same conditions.

50. The plant as claimed in any of claims 36 to 49, wherein water use efficiency is greater than in a control unmodified plant grown under the same conditions.

51. The plant as claimed in any of claims 36 to 49, wherein the enhanced photosynthesis results in one or more of the following traits: enhanced growth rate, reduced time to flowering, faster maturation, enhanced seed yield, enhanced biomass, increased plant height, and enhanced leaf canopy area, when compared to a control unmodified plant grown under the same conditions.

52. A plant part, plant tissue, plant organ, plant cell, plant protoplast, embryo, callus culture, pollen grain or seed, derived or obtained from the plant of any of claims 36 to 51.

53. A processed plant product obtained from the plant of any of claims 36 to 49 or the plant part, plant tissue, plant organ, plant cell, plant protoplast, embryo, callus culture, pollen grain or seed of claim 52; optionally wherein the processed product comprises a detectable nucleic acid sequence of (i) a phytochrome B or active fragment thereof downstream of a gene expression regulatory element active specifically in at least some of the vascular sheath cells of a plant, or (ii) at least a portion of a polynucleotide of any of claims 19 to 29.


SEQUENCE LISTING (10 sequences)

SEQ ID NO: 1 - DNA, 3519 bp, Artificial Sequence
  [YHB]domesticated
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggttcataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggcctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtcgcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagaatgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaggacggttcatttgtgctaaagagggaagagtttttccttggaagtgtcataaacgcgattgtaagtcaagcgatgttcttattaagggacagaggtctgcagctgatccgtgacattcccgaagagatcaaatcaatagaggtttttggagatcagataaggattcaacagctcctggctgagtttctgctgagtataatccggtatgcaccatctcaagagtgggtggagatccatttaagccaactttcaaagcaaatggctgatggattcgccgccatccgcacagaattcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattag

SEQ ID NO: 2 - DNA, 3519 bp, Arabidopsis thaliana
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacggttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattag

SEQ ID NO: 3 - DNA, 3516 bp, Oryza sativa
atggcctcgggtagccgcgccacgcccacgcgctccccctcctccgcgcggcccgcggcgccgcggcaccagcaccaccactcgcagtcctcgggcgggagcacgtcccgcgcgggagggggtggcgggggcgggggagggggagggggcggcgcggccgccgcggagtcggtgtccaaggccgtggcgcagtacaccctggacgcgcgcctccacgccgtgttcgagcagtcgggcgcgtcgggccgcagcttcgactacacgcagtcgctgcgtgcgtcgcccaccccgtcctccgagcagcagatcgccgcctacctctcccgcatccagcgcggcgggcacatacagcccttcggctgcacgctcgccgtcgccgacgactcctccttccgcctcctcgcctactccgagaacaccgccgacctgctcgacctgtcgccccaccactccgtcccctcgctcgactcctccgcggtgcctccccccgtctcgctcggcgcagacgcgcgcctccttttcgccccctcgtccgccgtcctcctcgagcgcgccttcgccgcgcgcgagatctcgctgctcaacccgctctggatccactccagggtctcctctaaacccttctacgccatcctccaccgcatcgatgtcggcgtcgtcatcgacctcgagcccgcccgcaccgaggatcctgcactctccatcgctggcgcagtccagtctcagaagctcgcggtccgtgccatctcccgcctccaggcgcttcccggcggtgacgtcaagctcctttgcgacaccgttgttgagtatgttagagagctcacaggttatgaccgcgttatggtgtacaggttccatgaggatgagcatggagaagtcgttgccgagagccggcgcaataaccttgagccctacatcgggttgcattatcctgctacagatatcccacaggcatcacgcttcctgttccggcagaaccgtgtgcggatgattgctgattgccatgctgcgccggtgagggtcatccaggatcctgcactaacacagccgctgtgcttggttgggtccacgctgcgttcgccgcatggttgccatgcgcagtatatggcgaacatgggttccattgcatctcttgttatggcagtgatcattagtagtggtggggatgatgatcataacatttcacggggcagcatcccgtcggcgatgaagttgtgggggttggtagtatgccaccacacatctccacggtgcatccctttcccactacggtatgcatgcgagttcctcatgcaagcctttgggttgcagctcaacatggagttgcagcttgcacaccaactgtcagagaaacacattctgcggacgcagacactgctgtgtgatatgctactccgggattcaccaactggcattgtcacacaaagccccagcatcatggaccttgtgaagtgtgatggtgctgctctgtattaccatgggaagtactaccctcttggtgtcactcccacagaagttcagattaaggacatcatcgagtggttgactatgtgccatggagactccacagggctcagcacagatagccttgctgatgcaggctaccctggtgctgctgcactaggagatgcagtgagtggaatggcggtagcatatatcacgccaagtgattatttgttttggttccggtcacacacagctaaggagataaagtggggtggtgcaaagcatcatccagaggataaggatgatggacaacgaatgcatccacgatcatcgttcaaggcatttcttgaagttgtgaagagtaggagcttaccatgggagaatgcggagatggatgcaatacattccttgcagctcatattgcgggactctttcagagattctgcagagggcacaagtaactcaaaagccatagtgaatggccaggttcagcttggggagctagaattacggggaatagatgagcttagctcggtagcaagggagatggttcggttgatcgagacagcaacagtacccatctttgcagtagatactgatggatgtataaatggttggaatgcaaaggttgctgagctgacaggcctctctgttgaggaagcaatgggcaaatcattggtaaatgatctcatcttcaaggaatctgaggaaacagtaaacaagctactctcacgagctttaagaggtgatgaagacaaaaatgtagagataaagttgaagacattcgggccagaacaatctaaaggaccaatattcgttattgtgaatgcttgttctagcagggattacactaaaaatattgttggtgtttgttttgttggccaagatgtcacaggacaaaaggtggtcatggataaatttatcaacatacaaggggattacaaggctatcgtacacaaccctaatcctctcatacccccaatatttgcttcagatgagaatacttgttgttcggagtggaacacagcaatggaaaaactcacaggatggtcaagaggggaagttgttggtaagcttctggtcggtgaggtctttggtaattgttgtcgactcaagggcccagatgcattaacgaaattcatgattgtcctacacaacgctataggaggacaggattgtgaaaagttccccttttcattttttgacaagaatgggaaatacgtgcaggccttattgactgcaaacacgaggagcagaatggatggtgaggccataggagccttctgtttcttgcagattgcaagtcctgaattacagcaagcctttgagattcagagacaccatgaaaagaagtgttatgcaaggatgaaggaattggcttacatttaccaggaaataaagaatcctctcaacggtatccgatttacaaactcgttattggagatgactgatctaaaggatgaccagaggcagtttcttgaaaccagcactgcttgtgagaaacagatgtccaaaattgttaaggatgctagcctccaaagtattgaggatggctctttggtgcttgagaaaggtgaattttcactaggtagtgttatgaatgctgttgtcagccaagtgatgatacagttgagagaaagagatttacaacttattcgagatatccctgatgaaattaaagaagcctcagcatatggtgaccaatatagaattcaacaagttttatgtgactttttgctaagcatggtgaggtttgctccagctgaaaatggctgggtggagatacaggtcagaccaaatataaaacaaaattctgatggaacagacacaatgcttttcctcttcaggtttgcctgtcctggcgaaggccttcccccagagattgttcaagacatgtttagtaactcccgctggacaacccaagagggtattggcctaagcatatgcaggaagatcctaaaattgatgggtggcgaggtccaatatataagggagtcggagcggagtttcttccatatcgtacttgagctgccccagcctcagcaagcagcaagtagggggacaagctga

SEQ ID NO: 4 - protein, 1172 aa, Artificial Sequence
  [YHB]domesticated
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVHKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 5 - protein, 1172 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 6 - protein, 1171 aa, Oryza sativa
MASGSRATPTRSPSSARPAAPRHQHHHSQSSGGSTSRAGGGGGGGGGGGGGAAAAESVSKAVAQYTLDARLHAVFEQSGASGRSFDYTQSLRASPTPSSEQQIAAYLSRIQRGGHIQPFGCTLAVADDSSFRLLAYSENTADLLDLSPHHSVPSLDSSAVPPPVSLGADARLLFAPSSAVLLERAFAAREISLLNPLWIHSRVSSKPFYAILHRIDVGVVIDLEPARTEDPALSIAGAVQSQKLAVRAISRLQALPGGDVKLLCDTVVEYVRELTGYDRVMVYRFHEDEHGEVVAESRRNNLEPYIGLHYPATDIPQASRFLFRQNRVRMIADCHAAPVRVIQDPALTQPLCLVGSTLRSPHGCHAQYMANMGSIASLVMAVIISSGGDDDHNISRGSIPSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTEVQIKDIIEWLTMCHGDSTGLSTDSLADAGYPGAAALGDAVSGMAVAYITPSDYLFWFRSHTAKEIKWGGAKHHPEDKDDGQRMHPRSSFKAFLEVVKSRSLPWENAEMDAIHSLQLILRDSFRDSAEGTSNSKAIVNGQVQLGELELRGIDELSSVAREMVRLIETATVPIFAVDTDGCINGWNAKVAELTGLSVEEAMGKSLVNDLIFKESEETVNKLLSRALRGDEDKNVEIKLKTFGPEQSKGPIFVIVNACSSRDYTKNIVGVCFVGQDVTGQKVVMDKFINIQGDYKAIVHNPNPLIPPIFASDENTCCSEWNTAMEKLTGWSRGEVVGKLLVGEVFGNCCRLKGPDALTKFMIVLHNAIGGQDCEKFPFSFFDKNGKYVQALLTANTRSRMDGEAIGAFCFLQIASPELQQAFEIQRHHEKKCYARMKELAYIYQEIKNPLNGIRFTNSLLEMTDLKDDQRQFLETSTACEKQMSKIVKDASLQSIEDGSLVLEKGEFSLGSVMNAVVSQVMIQLRERDLQLIRDIPDEIKEASAYGDQYRIQQVLCDFLLSMVRFAPAENGWVEIQVRPNIKQNSDGTDTMLFLFRFACPGEGLPPEIVQDMFSNSRWTTQEGIGLSICRKILKLMGGEVQYIRESERSFFHIVLELPQPQQAASRGTS

SEQ ID NO: 7 - DNA, 656 bp, Artificial Sequence
  nucleotide sequence of the vascular bundle promoter used
gatgataacacctgaatttaatgacaaaaaaaaaaaaaagtggatagagactagagggacagcaaggctgtgtgacatatatgggcagatagacaaagaagccgaaaaacgtgcaccgtccaagattctggctactatacctaatttccttcccgcagggacttgacaaatatcactatctgccatttttagttttattttgtattggtgtcaaagaattgaaataatgaacaacggtcgtaaaaagatgtaaatgtactgatgataacacctgaatttaatgacaaaaaaaaaaaaaagtggatagagactagagggacagcaaggctgtgtgacatatatgggcagatagacaaagaagccgaaaaacgtgcaccgtccaagattctggctactatacctaatttccttcccgcagggacttgacaaatatcactatctgccatttttagttttattttgtattggtgtcaaagaattgaaataatgaacaacggtcgtaaaaagatgtaaatgccattctagaagctactccacgtccataagggacacatcacaatcccactatccttcgcaagacccttcctctatataaggaagttcatttcatttggagaggacgacctgcaggtcgacggatccaaggagatataaca

SEQ ID NO: 8 - DNA, 5536 bp, Artificial Sequence
  Nucleotide sequence of the vascular bundle promoter used in the monocot vector
gaccggagtatatgtttatggaactttgtcatccatatcaagataactatacttctatttgttaaataataaatttaaaatatactgcttcaattaagggtgcacaataaaaatatacacgaaaaacgcggcaattgccgcgcagattttctagtacaaactaattactatcacaaaaggcttatttgtcatgtgtttaatgtgtttgagatggaacaggaattagtgacaaaattactttactcttgtccacttcacacaaattaccactcattttggatctcgtgagattaattggtatgatttcctatggtgtgttcttcgtgaacacaatagcttgcaatattggggtgcaaataggtatcatgtggtttgttgtgggtcatacacatctgtgtaactggatgatttactacgcgttatcgcggaaatttataataatgatgataatttattatggtaagatgaaaaaagctcaaaaaaataatcataatggatgaaaaaattaaaacatatgagaactatttaataaaattcatttgagctcattctgatgattaatcatatttgcttaaaaattcaaagttttttgactgcatttattaatgaaatttgaatataatattcaattatcatcagtagattggccaagttgcaagatctaaaacttcctagttaagattaaaaaatagtagatataacaacatcttaataaagtcgaggaaggtggagggtcagattgagcggaggtgcatgagaggtgaggattaaatagaggatagcagcgcgggagataaagaaggagggatgaaggaagttaaatggagaggtctacaatgaacggccatcattaggtagagcttcatgatcggacggtgcaaacaaaaaagatgatgtgactcgatgagaattcagttttgatagttagtgaatggtacaaatatatatagtacagataaatttgtgaatataagatggattttgatcattccagagagtttttgggtcagcccaaaatctaaaattagaaaaccaacatctctacctacaattggttaattgctatactaatttgttagtacgaggtgattttcttcgacttaaaaagcatttgcttacatggatgaagagagcgatgacatatacacaccctatatttggaacatgtagattcacttcaagtaaggaatcaattaacgcaaagtcatgacaaacaggattgaaacatacaacggttgttgtcagagaaagagagagagaaagtagtggtcgaagaaaaacccacaaccatgctcacaaaaagtctgaacctaacccgcaccgtcaccgccggatgggtcagaatgtaaccacggttaggctccggccagccctttaaaagcccgacggggctccgccgatacgcacccatacagcttgttccatttcatactcgtcgcgtacaccgcgacacagacagagcacgccgttctcctcgtacgtacgtgcctcttccttgtgcaagctcgatcgcagcgacgccctgctgctgctgctgctctctctccaagcgtcaatggcaggacgttaggatcgtgatcagtagggtgctaacgactctgtttccctgtcccgtttgcaggcggatcaacgaccaccgtgccgcacgcgcgccggcgagaatggatccgattcgttctcggacaccttctcccgctcgagagctccttcctggaccacaaccagatggagtgcaaccaacagctgataggggtgtatctcctccagccggtggacctcttgacggcttacctgcaaggcgcacaatgtcccgtaccagattgcccagtccaccagcaccaagtccagcgttttcagcgggcagcttctctgacctcctgagacagtttgatccctctttgttcaacacctcactgtttgactcacttccgccttttggggctcaccatactgaagccgctactggtgagtgggatgaggtgcagtcaggcttaagagcagctgatgccccaccacctacgatgagggtggcagtaactgctgctaggccaccgagagctaaaccagctccaagacgcagagcagcacaaccttccgatgcatctcctgctgctcaagtcgatcttcgcactttagggtatagccagcaacaacaggagaagatcaaacctaaggtccgaagtacagttgcgcaacaccatgaagcccttgttggtcatgggttcactcatgcccacatagttgcactatcccaacatcctgctgctcttggaactgttgcggtgaagtaccaggacatgattgctgctttacctgaagcaacacacgaggcaatagtcggtgttggcaaacagtggtctggcgctagggctctagaagccctccttacagttgcaggagaattgcggggacctcccttgcagctcgataccggacaattgctgaagattgccaaacgtggtggtgttacggcagtagaagcagtgcatgcttggaggaatgctctaactggagcacccttgaatctcacgccagaacaagtggtcgctatcgcctcccatgatggtggaaaacaagcactagaaactgtccaaagattattgcctgttctttgtcaggcacacggacttaccccacaacaagtcgttgctatagcctcccatgatggagggaagcaagcgttagaaacagtgcagcggctactccctgtattatgccaggctcatggtctaactccacaacaagtggtggctatagcctcacacgatggtggtaaacaggcacttgaaaccgtccaaagactcctgccggtcctctgccaggcacacggcctcacccccgaacaagtggtggctattgcttcgcatgatggaggtaagcaggctttagagacagtccagagactactacccgttctatgccaggcccatggtttgaccccggaacaggttgttgctattgcgtcaaataagggcggcaagcaagcgttggaaaccgttcaagcattactccctgttctctgtcaagcacatgggctaacgcccgagcaggttgttgcaattgcatcacatgatggaggaaagcaggccttagaaacggtacaggcacttttaccagtcctttgccaagcacacgggcttacacccgaacaagtggtcgctattgcaagtaatataggtggaaaacaagcactggaaaccgtgcaggcgcttttgccggtattatgccaagctcacggcctaactcctgaacaggtggttgcgattgcctcaaatggtgggggtaaacaggcactggagactgtgcagcggcttttgcctgttttgtgtcaagctcatggattgacaccagagcaggtggtcgctatagctagtaacattggaggtaaacaagcgcttgaaaccgtgcaacgtctgctgccagttctatgtcaagctcatgggttgaccccacaacaggttgtagcgatcgcttccaataaaggaggaaagcaagctctagaaacggtgcagaggctcctcccggttctttgtcaggcgcatggattgaccccggagcaggtggtcgcaatcgccagtcatgatggaggtaagcaggccttggaaaccgttcaggcgttactcccggttctatgccaggcgcatggcctgacccctgaacaggttgtggcgatagccagtaacggcgggggaaagcaggcacttgaaaccgtacaacgactcctcccagtcctttgtcaagcccacggattgactccagaacaagtagttgctatagcttcgaataagggaggaaagcaggcccttgaaacagttcagcgtcttttgccagtgttgtgtcaagcacacggattgactcctgaacaggttgtcgccattgcatctaatatcggtggtaagcaagctctcgaaaccgtacagcgactcttgcctgttctatgccaagcgcatggcttgacgccggaacaggtggtagccatagcaagcaacataggtggcaaacaagctcttgaaacagttcaaaggttgttacctgtgctttgccaagcccacggtttgacccctcaacaggtggttgctatagcatcacatgatgggggacggcctgctcttgagacagtgcagcgcctgttgcccgtgttgtgtcaagcgcatggcttaacaccggaacaggtcgtggcaattgcgtcaaatattggcggcaaacaagcgctggaaaccgttcagcgactcttgcctgttctgtgccaagctcacggtctgacgccccaacaggttgttgccattgcttcaaatggaggagggaggccagcccttgagtcgattgtcgcacagctatctcggcccgaccctgctttagccgctctgacaaatgatcatcttgtggctctcgcctgcttaggaggtcgcccagctttagacgcagtaaaaaagggtctacctcatgctccggccttaatcaagaggacgaatcgtagaatcccagaacgaacgagccatcgcgtagccgatcacgctcaagttgttagggttttaggtttttttcagtgtcattcacatccggcacaagctttcgatgatgccatgacccagtttggtatgtcaaggcatggattactgcaacttttcagaagagtaggagtgacagagctcgaagccagaagcggaactctgccacccgctagccaaagatgggataggatattgcaggcgagtggaatgaagcgcgcgaaaccatctccaacaagcactcaaaccccggatcaagcgagtttgcacgctttcgcagattctctcgaacgagatttggatgccccttctccaatgcacgaaggtgatcaaactagggcgagtagcaggaagaggtctaggagtgatcgtgcagttacgggcccctcagcacaacagtcttttgaggtcagggtgccagaacaaagggacgctttacatctcccattgtcttggcgtgtaaaaaggccgcgaactagtattggagggggattaccggacccagggacccccactgctgctgatctagctgcttctagtacggtaatgcgcgagcaagacgaggatccatttgctggggcagctgatgacttccccgcattcaacgaagaagaattagcatggttgatggagttactgccacagtaagcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaattcgagctcggaggttatcacacttggtaatttccccgcatagctgaacatctatacaacactcatcgcgcggagaaacgcgcaacaaattggaggcgcatt

SEQ ID NO: 9 - DNA, 3495 bp, Arabidopsis thaliana
atggtctccggaggtggtagcaaaaccagcggtggagaggcagcttcctcaggccatcgccgaagtcgtcacaccagcgctgcagaacaagctcagtcgtcagcaaacaaagccctaaggtcacagaatcagcagccacaaaaccacggtggcggaacagagtccacaaacaaagctattcaacagtacactgtcgacgcgagactccacgccgtcttcgaacaatccggagagtcaggtaagtcgtttgattactcacagtctcttaaaacggcgccgtacgattcctccgtaccagagcagcagatcacagcttatctctcccggatccaacgcggtggctatacccagccttttggctgcttgatcgccgtcgaagaatccactttcacaatcatcggttacagtgaaaatgcgcgggaaatgctagggctcatgtctcaatctgtaccaagcatcgaggacaaatcagaggttttaacgattggtacggatttgcgatctctcttcaagtcatcgagctaccttctcctcgagcgcgcgttcgtggctcgagagatcacgcttctgaatcctatttggattcactctaacaacactggtaaacctttctacgcgattctccacagggttgatgttggaattttgatcgatttagagccggctcgaaccgaagatccggcactttcaatcgccggagcagtccaatcgcagaaacttgcggtacgtgcgatttctcatttacaatcgttgcctagcggcgacattaagcttctatgtgacactgttgtggaaagcgttagagatcttactggctacgaccgcgttatggtgtacaagtttcatgaagatgaacatggtgaagtcgtagccgagagtaaacggaacgatttagagccttacattggtctgcattatcccgctactgatattcctcaggcatctcggttcttgttcaagcaaaaccgtgttaggatgatagtagattgctatgcgtcaccggttcgtgtggttcaagacgataggctcacgcagtttatatgcttggtgggttcgactttgcgagctcctcatggctgtcatgctcaatacatgactaacatgggctctattgcgtcgttagctatggcagttataataaatggaaacgaagaagatggtaatggggttaatactggaggaagaaactcgatgaggctttggggtttagttgtttgccatcacacatcagctcgttgcataccttttcctttgaggtacgcttgtgagtttcttatgcaggcctttggcttacagctaaacatggagttgcagttagccttgcaggtgtctgaaaaacgcgttctgagaatgcagacactattatgtgatatgcttctacgtgactcaccagcggggattgtcacgcagaggcctagtatcatggatttagtaaaatgtaatggtgcggcatttctttaccaagggaagtattatccgttgggtgtgactccaactgattctcagattaatgacattgtggagtggttggttgctaaccattctgattctaccgggttaagcacagatagtttaggcgatgcgggttatcctcgggcagctgctttgggagatgctgtgtgcggtatggcagtcgcgtgtatcacaaaaagggacttccttttctggtttcggtctcatactgagaaagaaatcaaatggggaggggctaagcaccatcctgaggacaaagatgatggtcagcggatgaatccgcgttcttcgttccagacttttctcgaagttgttaagagccgatgtcagccatgggaaactgctgaaatggacgccattcactcgctccagcttattctaagagactctttcaaagagtctgaagcgatggactctaaagctgctgcagctggggcggttcagccacatggagatgatatggtacagcaagggatgcaggagataggtgcagttgcaagagagatggttaggctcattgagactgcgacggttcctatatttgctgtggacatagacggttgcatcaatgggtggaacgccaagatcgcagagctgaccggtctttctgttgaagacgctatgggaaagtcgctggttcgcgaattgatatacaaagagtacaaagaaacagttgataggcttctttcttgtgctctcaaaggggatgaaggcaagaatgtggaggtcaagctgaaaacttttggttccgagctacaaggaaaagcaatgtttgtggttgtcaacgcatgttcaagcaaggactacttaaacaacatcgttggagtctgctttgttggacaagatgtaactggtcataaaattgttatggacaagttcatcaacatacaaggtgattacaaggccatcatccatagcccgaaccctctgatccctccaatctttgcagcggatgagaatacgtgctgccttgagtggaacactgcaatggaaaagctcacaggctggcctcgcagcgaagtgattggaaaattacttgttagggaagtatttgggagctattgcagactaaagggtcctgatgcgttaactaagttcatgatcgtcttgcataacgcgatcggtggccaagatactgataaattcccattcccgttctttgatcgcaaaggggaattcattcaggctctcctgactttgaacaaacgggtcagcatcgatggcaaaatcattggggctttctgttttttgcagataccgagtcccgagctgcagcaagctctagaagttcagaggaggcaggagagtgaatatttctcaaggaggaaagagttggcttacattttccaagttataaagaatccattgagtggattgcgtttcacaaattcattgctggaagacatggatttaaacgaggatcagaagcagcttcttgaaacgagtgtttcatgtgagaagcagatctcaaagattgtaggagacatggacgtcaaaagcatagatgacggttcatttctgctagagagaacagagttcttcattggcaatgtcacaaatgcagtggtaagccaagtcatgttggtggtgagagagagaaatctccagctgatccgtaacattcccacggaggtcaaatccatggctgtctacggtgaccagataaggctccaacaggttctcgcagaatttctgctaagtattgtccgttatgcacccatggaaggctcggtagagctccatctatgcccgactctgaaccaaatggctgacggattctccgctgtacgtttggagttcagaatggcgtgtgcaggggaaggtgtgccgccagagaaagtgcaagacatgttccatagtagccgatggacaagtccagaaggattaggactaagcgtttgcagaaagattttgaagctgatgaacggaggggttcagtacataagagaattcgaacgctcttatttcctaatcgttatcgaactcccggttcctctaatgatgatgatgccctcttcatga

SEQ ID NO: 10 - protein, 1164 aa, Arabidopsis thaliana
MVSGGGSKTSGGEAASSGHRRSRHTSAAEQAQSSANKALRSQNQQPQNHGGGTESTNKAIQQYTVDARLHAVFEQSGESGKSFDYSQSLKTAPYDSSVPEQQITAYLSRIQRGGYTQPFGCLIAVEESTFTIIGYSENAREMLGLMSQSVPSIEDKSEVLTIGTDLRSLFKSSSYLLLERAFVAREITLLNPIWIHSNNTGKPFYAILHRVDVGILIDLEPARTEDPALSIAGAVQSQKLAVRAISHLQSLPSGDIKLLCDTVVESVRDLTGYDRVMVYKFHEDEHGEVVAESKRNDLEPYIGLHYPATDIPQASRFLFKQNRVRMIVDCYASPVRVVQDDRLTQFICLVGSTLRAPHGCHAQYMTNMGSIASLAMAVIINGNEEDGNGVNTGGRNSMRLWGLVVCHHTSARCIPFPLRYACEFLMQAFGLQLNMELQLALQVSEKRVLRMQTLLCDMLLRDSPAGIVTQRPSIMDLVKCNGAAFLYQGKYYPLGVTPTDSQINDIVEWLVANHSDSTGLSTDSLGDAGYPRAAALGDAVCGMAVACITKRDFLFWFRSHTEKEIKWGGAKHHPEDKDDGQRMNPRSSFQTFLEVVKSRCQPWETAEMDAIHSLQLILRDSFKESEAMDSKAAAAGAVQPHGDDMVQQGMQEIGAVAREMVRLIETATVPIFAVDIDGCINGWNAKIAELTGLSVEDAMGKSLVRELIYKEYKETVDRLLSCALKGDEGKNVEVKLKTFGSELQGKAMFVVVNACSSKDYLNNIVGVCFVGQDVTGHKIVMDKFINIQGDYKAIIHSPNPLIPPIFAADENTCCLEWNTAMEKLTGWPRSEVIGKLLVREVFGSYCRLKGPDALTKFMIVLHNAIGGQDTDKFPFPFFDRKGEFIQALLTLNKRVSIDGKIIGAFCFLQIPSPELQQALEVQRRQESEYFSRRKELAYIFQVIKNPLSGLRFTNSLLEDMDLNEDQKQLLETSVSCEKQISKIVGDMDVKSIDDGSFLLERTEFFIGNVTNAVVSQVMLVVRERNLQLIRNIPTEVKSMAVYGDQIRLQQVLAEFLLSIVRYAPMEGSVELHLCPTLNQMADGFSAVRLEFRMACAGEGVPPEKVQDMFHSSRWTSPEGLGLSVCRKILKLMNGGVQYIREFERSYFLIVIELPVPLMMMMPSS